(19) United States
(12) Patent Application Publication
(10) Pub. No.: US 2010/0203114 A1
(43) Pub. Date: Aug. 12, 2010

(54) MICELLE ENCAPSULATION OF THERAPEUTIC AGENTS

(75) Inventors: Glen S. Kwon, Waunakee, WI (US); M. Laird Forrest, Lawrence, KS (US); Neal M. Davies, Pullman, WA (US)

Correspondence Address: Schwegman Lundberg & Woessner/WARF, P.O. Box 2938, Minneapolis, MN, 55402, US

(73) Assignee: WARF - Wisconsin Alumni Research Foundation, Madison, WI (US)

(21) Appl. No.: 12/668,269

(22) PCT Filed: Jul. 9, 2008
(86) PCT No.: PCT/US08/08417

§ 371 Date: Apr. 13, 2010

Related U.S. Application Data
(60) Provisional application No. 60/948,642, filed on Jul. 9, 2007.
(60) Provisional application No. 61/044,813, filed on Apr. 14, 2008.

(51) Int. Cl. A61K 31/33; A61K 31/337; A61K 31/7048; A61P 35/00; A61P 35/02; A61K 9/127

(52) U.S. Cl. 424/450; 514/183; 514/449; 514/27

(57) ABSTRACT

The invention provides 17 AAG encapsulated in micelle particles. The micelles can be comprised of safe poly(ethylene glycol)-block-poly(lactic acid) (PEG-PLA). A significant advantage of PEG-PLA as a carrier is that it is less toxic than Cremophor® EL or DMSO. Additionally, PEG-PLA micelles are easier to handle than DMSO and they do not possess foul odors, which is a problem associated with 17-AAG formulations currently in clinical trials. The invention also provides methods of preparing active agents encapsulated micelles and therapeutic methods of using the micelles and their corresponding formulations, such as for the inhibition of Hsp 90, and/or for the treatment of cancer.

### RELATED APPLICATIONS

This application claims priority under 35 U.S.C. §119(e) to U.S. Provisional Patent Application Nos. 60/948,642, filed Jul. 9, 2007, and 61/044,813, filed Apr. 14, 2008, which are incorporated herein by reference.

### GOVERNMENT SUPPORT

This invention was made with government support under Contract No. AI043346 awarded by the National Institutes of Health. The United States Government has certain rights in the invention.

### BACKGROUND OF THE INVENTION

Heat Shock Protein 90 (Hsp90) is an important target for cancer therapy due to its key role in regulating proteins that are involved in tumor cell proliferation. It was discovered that geldanamycin, a benzoquinone ansamycin antibiotic, can strongly bind to the ATP/ADP binding pocket of Hsp90, interfering with the survival and growth of a diverse family of tumors. Geldanamycin is a promising new anticancer agent, but its clinical development has been hampered by severe hepatotoxicity and poor solubility. Two analogues, 17-allylamino-17-demethoxygeldanamycin (17-AAG; tanespimycin) and 17-dimethylamino-ethylamino-17-demethoxygeldanamycin (17-DMAG), were developed to alleviate these issues.

Several promising leads for clinical translation have been directed to development of 17-DMAG as the more pharmaceutically practical formulation because 17-DMAG possesses superior aqueous solubility and greater oral bioavailability compared to 17-AAG. However, despite its apparent advantages over 17-AAG, 17-DMAG is characterized by a large volume of distribution when administered to animals. This wide distribution could lead to undesired toxicity because the maximum tolerated dose of 17-DMAG is significantly less than that of 17-AAG (8 mg/m²/day and 100-200 mg/m²/day in dogs, respectively).

The major obstacle for delivery of 17-AAG is its limited aqueous solubility (about 0.1 mg/mL), which has resulted in the use of complicated formulations with Cremophor® EL (CrEL), DMSO, and/or ethanol before parenteral administration. This is undesirable from a patient tolerability standpoint because CrEL is known to induce hypersensitivity reactions and anaphylaxis, and requires patient treatment with antihistamines and steroids before administration.

Therefore, safer and more effective delivery of 17-AAG relies on the development of biocompatible delivery systems capable of solubilizing the drug without the use of harsh surfactants. Accordingly, there is a need for improved compositions for the delivery of 17-AAG to patients.

### SUMMARY

The invention provides active agents, such as 17-AAG (17-allylamino-17-demethoxygeldanamycin), encapsulated by safe polyethylene glycol)-block-poly(lactic acid) (“PEG-PLA”) micelles. The compositions of the invention therefore provide effective solubilization of the active agents. A significant advantage of PEG-PLA as a carrier is that it is less toxic than Cremophor® EL or DMSO, which are used in currently known compositions. Additionally, PEG-PLA micelles are easier to handle than DMSO and they do not possess a foul odor, which is a problem with 17-AAG formulations currently in clinical trials. Micelle encapsulation can also reduce the occurrence of side effects (e.g., hepatotoxicity, neutropenia, neuropathy, and the like) of certain agents by maintaining the agents within the micelles until they are delivered to the target area of the body.

The PEG-PLA micelles can be used to encapsulate 17-AAG and/or a second active agent to provide a pharmaceutical composition. The second active agent can be a chemotherapeutic agent, such as paclitaxel, docetaxel, teniposide, or etoposide. The composition can be formulated to be suitable for various forms of internal administration, such as intravenous (IV) injection or infusion. Such formulations can include 17-AAG-containing micelles that have a 17-AAG concentration of up to about 20 mg/mL. The formulations can include a suitable aqueous carrier, such as a saline or dextrose solution.

The invention also provides a method of administering 17-AAG to a patient in need thereof, comprising administering an effective amount of a composition that includes 17-AAG encapsulated within micelles. The micelles can comprise block copolymers that include one or more blocks of poly(ethylene glycol) and polylactic acid).

The invention further provides a method of killing cancer cells by contacting the cells with a composition that includes an effective amount of 17-AAG-containing micelles. The contacting can be in vivo or in vitro. The 17-AAG containing micelles can also be used to prevent, slow, or inhibit the growth of a cancer tumor by contacting, or administering to, the tumor a composition that includes an effective amount of 17-AAG-containing micelles.

The invention further provides a method of preparing 17-AAG-containing micelles, wherein 17-AAG and unimers (block polymer chains not in micellar arrangement) are first dissolved in a water-miscible solvent (for example, dimethylacetamide (DMAc)). The solution can then be added to a dialysis apparatus (e.g., a bag or tubing). The dialysis apparatus can then be placed in an aqueous bath, to provide the 17-AAG-containing micelles in the dialysis bag. The micelles can then be separated, for example, by centrifugation to precipitate unincorporated drug. Nano-filtration can be used to provide the isolated 17-AAG-containing micelles.

The invention also provides a method of preparing 17-AAG-containing micelles where 17-AAG and PEG-PLA (2000 mw; 50:50) are dissolved in a suitable solvent, such as acetonitrile or DMAc. In one embodiment, the blocks of the PEG-PLA can be an approximate 50:50 of 2,000 mw blocks. The solution can be sonicated and then concentrated by solvent removal. Warm water (50-70° C., or approximately 60° C.) can then be added and the mixture can be allowed to cool to ambient temperature (˜23° C.). The mixture can then be separated, for example, by centrifugion to remove sediment (e.g., at about 13,000 rpm for about one minute). The supernatant can be collected and filtered, for example, through a 0.2 μm PTFE filter, to provide the isolated 17-AAG-containing micelle formulation. The resulting micelle pharmaceutical solution can be stored at low temperatures, e.g., about 4° C., for extended periods of time.

### BRIEF DESCRIPTION OF THE DRAWINGS

The following drawings form part of the present specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention, however, one skilled in the art will understand that portions of the example or aspect may be used in combination with other examples or aspects of the invention.

FIG. 1 illustrates characterization of micelles, according to an embodiment of the invention; (a) dynamic light scattering (DLS) of unloaded micelles revealed particles with average diameters of 242±5 nm; and (b) the critical micelle concentration of PEG-PLA (12:6 kDa) determined as 350 nM.

FIG. 2 illustrates in vitro release of 17-AAG from 0.3-mM PEG-PLA micelles in ddH₂O at 37° C. and pH 7.4, according to one embodiment. Micelles were prepared with 11.4% w/w 17-AAG based on PEG-PLA content, as noted in Table 1; PEG-PLA drug-loaded micelles (); drug alone (∇); and 17-AAG formulated in CrEL-EtOH-PEG400 (▪).

FIG. 3 illustrates pharmacokinetic profiles in rats, including (a) serum, (b) amount of 17-AAG remaining in the urine, (c) rate of urine excretion of 17-AAG, and (d) total amount of 17-AAG excreted through the urine, according to one embodiment. Each administered formulation included 10 mg/kg of 17-AAG (n=10, mean±SEM).

FIG. 4 illustrates (a) Tissue Distribution of 17-AAG in rats after injection, 3 hours post dose (10 mg/kg; n=10, mean±SEM); and (b) Tissue to Serum Ratio (Kp) following intravenous administration of 17-AAG (10 mg/kg) to rats, 3 hours post dose (n=10, mean±SEM). The asterisk (*) denotes statistically significant differences (p<0.05) between the standard formulation of CrEL-EtOH-PEG400 and 17-AAG encapsulated in PEG-PLA micelles.

FIGS. 5 (a) and 5(b) illustrate 17-AAG micelle preparation procedures according to embodiments of the invention.

FIG. 6 illustrates a characterization of the drug loading of PEG-PLA micelles encapsulating 17-AAG, determined by UV measurements at 337 nm, according to an embodiment.

FIG. 7 illustrates particle size distribution in PEG-PLA micelles determined by DLS measurement at 632.8 nm, according to one embodiment; (a) number-weight Gaussian distribution, and (b) volume-weight Gaussian distribution.

FIG. 8 shows a transmission electron microscopy (TEM) image of PEO-b-PDLA micelles (18,000×).

FIG. 9 illustrates a micelle loading and formation procedure, according to one embodiment. After nanofiltration the formulation can be analyzed using HPLC with UV and RI detection modes.

FIG. 10 illustrates the solubilization of paclitaxel and 17-AAG together in PEG-PLA micelles.

FIG. 11 illustrates physical stability results for 17-AAG/paclitaxel PEG-PLA micelles.

FIG. 12 illustrates (a) solubility of paclitaxel (PTX) and 17-AAG dual agent micelles (initial); (b) solubility of paclitaxel and 17-AAG dual agent micelles after 24 hours; (c) a comparison of paclitaxel concentration in PTX/17-AAG dual agent micelles at 0 hours and at 24 hours; and (d) a comparison of paclitaxel concentration in paclitaxel-only micelles at 0 hours and at 24 hours.

FIG. 13 illustrates (a) solubility of etoposide (ETO) and 17-AAG dual agent micelles (initial); (b) solubility of etoposide and 17-AAG dual agent micelles after 24 hours; (c) a comparison of etoposide concentration in ETO/17-AAG dual agent micelles at 0 hours and at 24 hours; and (d) a comparison of etoposide concentration in etoposide-only micelles at 0 hours and at 24 hours.

FIG. 14 illustrates (a) solubility of docetaxel (DCTX) and 17-AAG dual agent micelles (initial); (b) solubility of docetaxel and 17-AAG dual agent micelles after 24 hours; (c) a comparison of docetaxel concentration in DCTX/17-AAG dual agent micelles at 0 hours and at 24 hours; and (d) a comparison of docetaxel concentration in docetaxel-only micelles at 0 hours and at 24 hours.

FIG. 15 illustrates (a) the physical stability of paclitaxel (PTX) single agent micelles over time, as analyzed by optical density (OD) changes; and (b) the physical stability of paclitaxel/17-AAG dual agent micelles over time, as analyzed by optical density (OD) changes.

### DETAILED DESCRIPTION

Geldanamycin is a well-known natural product, obtainable by culturing the producing organism, Streptomyces hygroscopicus var. geldanus NRRL 3602. The compound 17-AAG is made semi-synthetically from geldanamycin, by reaction of geldanamycin with allylamine, as described in U.S. Pat. No. 4,261,989 (Sasaki et al.), the disclosure of which is incorporated herein by reference.

Geldanamycin binds strongly to the ATP/ADP binding pocket of Hsp90, thus interfering with the survival and proliferation of a diverse family of tumors, including HER-2/erbB-2 overexpressing, paclitaxel resistant breast cancers. Clinical development of geldanamycin has been hampered by its poor solubility and severe hepatotoxicity (Ge et al., J. Med. Chem. 49(15) (2006) 4606-4615). Thus, a significant obstacle in the preparation of pharmaceutical formulations containing geldanamycin, or its derivatives such 17-allylamino-17-demethoxy-geldanamycin (17-AAG, Scheme 1 below), is the very poor water solubility of these lipophilic drugs.

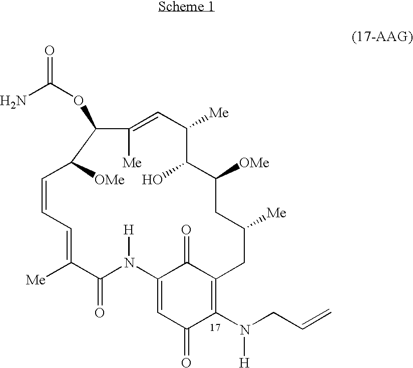

Suitable water solubility is of particular importance for parenteral administration. The water solubility of 17-AAG is only about 0.1 mg/mL at neutral pH, making it difficult to administer in a safe and effective manner. Attempts have been made to address the solubility issue but each formulation was accompanied by its own drawbacks, such as the use of DMSO, ethanol, or various undesirable surfactants.

The compound 17-AAG (17-all ylamino-17-demethoxygeldanamycin, or tanespimycin) is a promising heat shock protein 90 inhibitor currently undergoing clinical trials for the treatment of cancer. Despite its selective mechanism of action on cancer cells, 17-AAG faces challenging issues due to its poor aqueous solubility. Current 17-AAG compositions require formulation with Cremophor® EL (CrEL), DMSO, and/or ethanol. See U.S. Application Publication No. 2005/0256097 (Thong et al.).

Cremophor® EL is a castor oil derivative, typically prepared by reacting 35 moles of ethylene oxide with each mole of castor oil to provide a polyethoxylated castor oil (CAS number 61791-12-6). The use of Cremophor® EL (e.g. KOS-953) or DMSO for parenteral formulations is undesirable from a patient tolerability standpoint due to its potential side effects. Various adverse effects can include acute hypersensitivity reactions, peripheral neurotoxicity, hyperlipidaemia, and/or inhibition of P-glycoprotein. Furthermore, 17-AAG has a high volume of distribution (Vd) and considerable systemic toxicity at low doses (less than 20 mg/kg). Accordingly, improved formulations are needed to safely administer 17-AAG to patients in need of such treatment.

The disclosure herein provides a CrEL-free formulation of 17-AAG, prepared using amphiphilic diblock micelles composed of poly(ethylene oxide)-b-poly(D,L-lactic acid) (PEG-PLA). Dynamic light scattering (DLS) revealed PEG-PLA (12:6 kDa) micelles with average diameters of about 257 nm and critical micelle concentration of about 350 nM. The micelles can solubilize significant quantities of certain active agents, for example, about 1.5 mg/mL of 17-AAG. The area under the curve (AUC) of PEG-PLA micelles was 1.3-fold that of the standard formulation. Renal clearance (CLᵣₑₙₐₗ) increased and hepatic clearance (CL_{hepatic}) decreased with use of the micelle formulation, as compared to the standard vehicle that employs CrEL. Accordingly, the micelle formulations described herein provide delivery vehicles for 17-AAG that have several advantages over currently known compositions.

Additionally, the 17-AAG micellar formulation showed a 2.7-fold increase in the half-life (t_{1/2}) of the drug in serum and 1.3-fold increase in t_{1/2} in urine. As expected, because the 17-AAG circulated for a longer period of time in the blood, a 1.7-fold increase in the volume of distribution (V_{d}) was also observed with this micelle formulation, due to non-specificity (no targeting moiety) compared to the more rapidly cleared standard formulation. The new formulations of 17-AAG in PEG-PLA (12:6 kDa) micelles resulted in a favorable 150-fold increase in solubility over 17-AAG alone. This data indicates that the nanocarrier system of the invention can retain the pharmacokinetic disposition of 17-AAG without the need for toxic agents such as CrEL and EtOH.

The term PEG-PLA refers to poly(ethylene oxide)-block-poly(lactic acid). The poly(lactic acid) block can include (D-lactic acid), (L-lactic acid), (D,L-lactic acid), or combinations thereof. Various forms of PEG-PLA are available commercially, such as from Polymer Source, Inc., Montreal, Quebec, or they can be prepared according to methods well known to those of skill in the art. The molecular weight of the poly(ethylene glycol) block can be about 1,000 to about 35,000 g/mol, or any increment of about 500 g/mol within said range. Suitable blocks of the poly(lactic acid) can have molecular weights of about 1,000 to about 15,000 g/mol, or any increment of about 500 g/mol within said range. The PEG block can terminate in an alkyl group, such as a methyl group (e.g., a methoxy ether) or any suitable protecting, capping, or blocking group.

It should be noted that references in the specification to “one embodiment”, “an embodiment”, “an example embodiment”, etc., indicate that the embodiment described may include a particular feature, structure, or characteristic, but every embodiment may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

### Methods of Preparing PEG-PLA Micelles

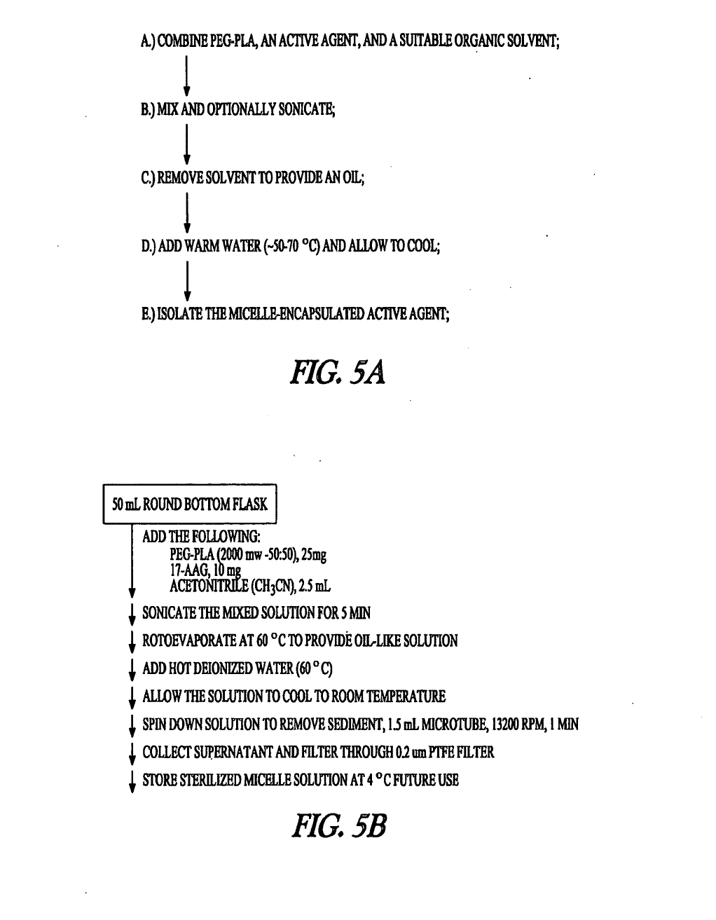

Amphiphilic unimers (single chains) present in a solvent in an amount above the critical micelle concentration (CMC) aggregate into a micelle, a core-coronal structure with a hydrophobic interior and hydrophilic exterior, or shell. PEG-PLA micelles can be prepared as described in Example 1, as well as schematically illustrated in FIG. 5(a). FIG. 5(b) provides one specific procedure for preparing a 17-AAG micelle formulation. This procedure is merely illustrative for one embodiment and the procedure can be varied according to the desired scale of preparation, as would be readily recognized by one skilled in the art. One advantage of the procedure illustrated in FIG. 5 is that it does not require dialysis of a micelle solution.

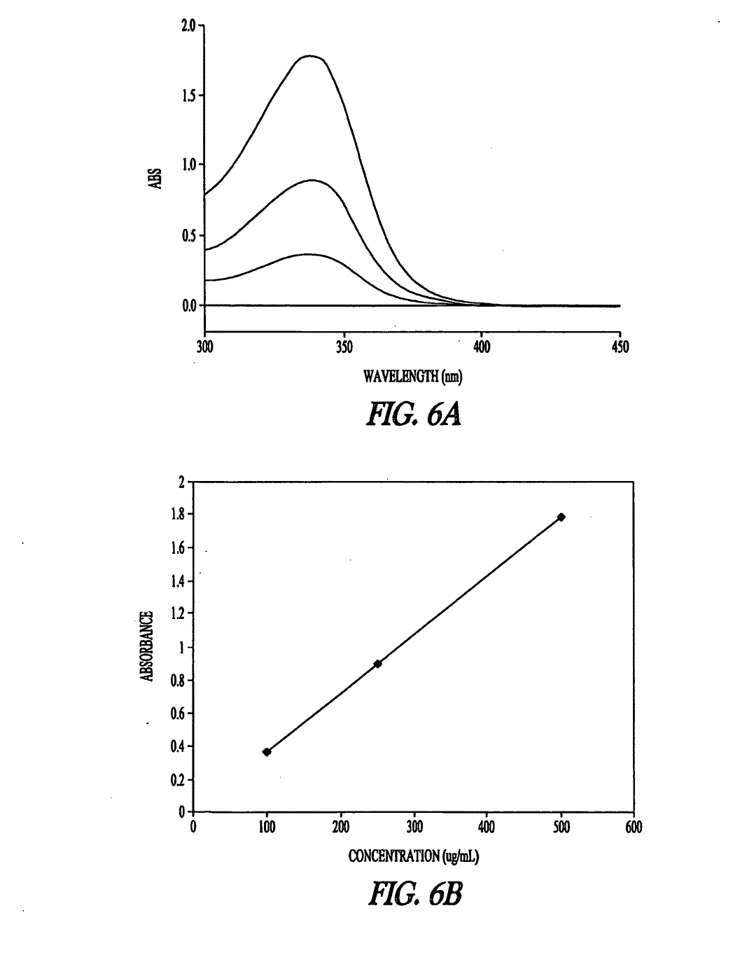

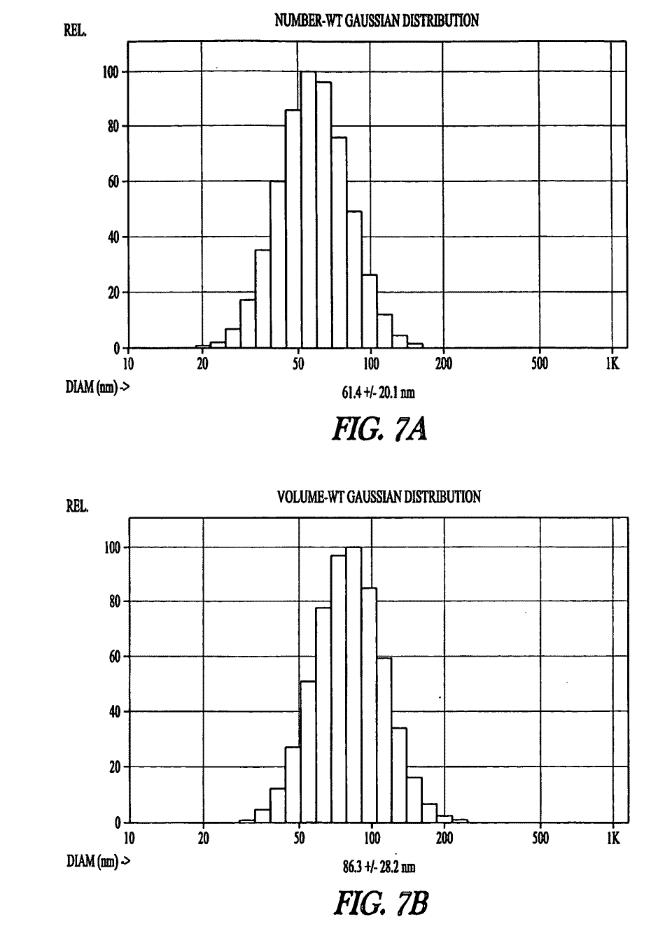

The micelles of this disclosure can be prepared using PEG-PLA polymers of a variety of block sizes (e.g., a block size within a range described above) and in a variety of ratios (e.g., PEG:PLA of about 1:10 to about 10:1, or any integer ratio within said range). For example, molecular weights (Mₙ) of the PEG-PLA polymers can include, but are not limited to, 2K-2K, 3K-5K, 5K-3K, 5K-6K, 6K-5K, 6K-6K, 8K-4K, 4K-8K, 12K-3K, 3K-12K, 12K-6K, and/or 6K-12K. The drug-to-polymer ratio can also be about 1:20 to about 10:1, or any integer ratio within said range. Specific examples of suitable drug-polymer ratios include, but are not limited to, about 1:2.5; about 1:5; about 1:7.5; and/or about 1:10. One suitable polymer is a PEG-PLA that includes blocks of about 1-3 kDa (e.g., about 2K Daltons) at an approximate 50:50 ratio. Use of this procedure resulted in unexpectedly high levels of drug loading in the micelles. For example, when the procedure of FIG. 5 was employed, drug loading of about 5 mg/mL was achieved (about 9 mM; about 17 wt %) (see FIG. 6).

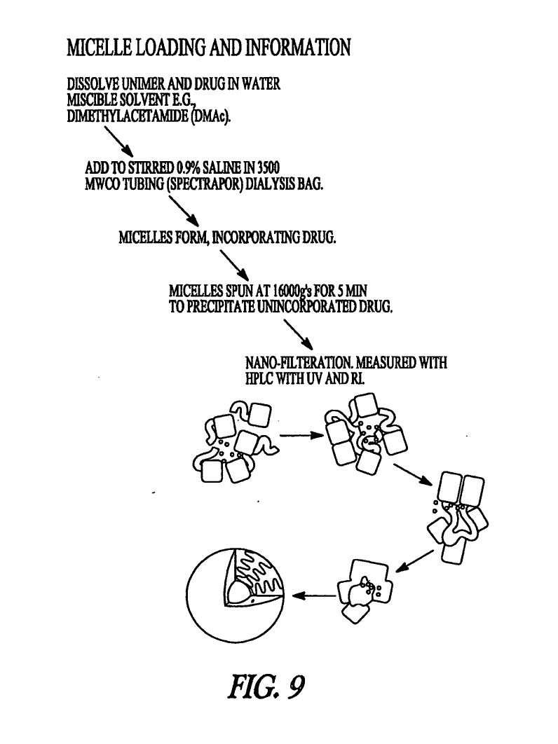

A micelle-encapsulated active agent formulation can also be prepared by a dialysis method. The agent and the PEG-PLA can be dissolved in a suitable water miscible organic solvent, such as dimethylacetamide (DMAc). The solution is then transferred to a dialysis bag. The dialysis medium contains an aqueous solution, such as 0.9% saline. The dialysis bag can be, for example, a 3500 MWCO tubing (SpectraPor®) dialysis bag. Upon addition of the dissolved agent and polymer to the aqueous mixture, micelles form, incorporating the active agent. The micelle-encapsulated drugs can then be isolated. For example, unincorporated drug can be precipitated by centrifugation. Nanofiltration of the resulting supernatant provides the isolated micelle-encapsulated active agent formulation. See FIG. 9. The micelles can be measured and analyzed, for example, using an HPLC equipped with UV and RI detection modes (see the techniques described by Yasugi et al., J. Control. Release, 1999, 62, 99-100). Preparatory methods can also include the use of oil-in-water emulsions, solution casting, and/or freeze-drying (lyophilization). See Gaucher et al., J. Controlled Release, 109 (2005) 169-188.

Once prepared, the micelle-drug composition can be stored for extended periods of time under refrigeration, preferably at a temperature between about −20° C. and about 4° C. Use of brown glass vials or other suitable containers to protect the micelle-drug composition from light can extend their effective lifetime. The micelle-drug compositions can be freeze-dried into a solid formulation, which can be reconstituted with an aqueous vehicle prior to drug administration.

### Formulations

By incorporating 17-AAG into PEG-PLA micelles, a larger amount of the drug can be dissolved in a given amount of fluid, such as a pharmaceutical carrier, or body fluid, such as blood or interstitial fluids, than can be dissolved without use of the micelles. Thus, the micelle effectively solubilize the 17-AAG to a higher degree than would be otherwise possible. A pharmaceutical carrier that dissolves the micelles such that the micelles can pass through a filter are considered to be dissolved in a pharmaceutical “solution”, to provide a formulation according to an embodiment of the invention.

In one embodiment, the micelles can solubilize up to about 15 mg/mL of 17-AAG, or up to about 20 mg/mL of 17-AAG. In some embodiments, the micelles can solubilize about 3 mg/mL, about 5 mg/mL, about 10 mg/mL, about 15 mg/mL, about 20 mg/mL, or about 25 mg/mL of an active agent. In some embodiments, the formulation can have concentrations of about 0.5 to about 5 mg/mL of 17-AAG, about 0.75 to about 3 mg/mL of 17-AAG, about 1 to about 2 mg/mL of 17-AAG, or about 1.5 mg/mL, with respect to the volume of micelles or preferably, the volume of the aqueous carrier. Similar amounts of other active agents can be included in micelles of certain other embodiments.

In one embodiment, 17-AAG encapsulated micelles are formulated in a mixture that includes an aqueous carrier, such as saline or dextrose, and the like. For example, a suitable carrier can be 0.9% NaCl solution, or a 5% aqueous saccharide solution, such as a dextrose or glucose solution. See, Remington: The Science and Practice of Pharmacy, D. B. Troy, Ed., Lippincott Williams & Wilkins (21^{st} Ed., 2005) at pages 803-849.

For purposes of administration, for example, parenteral administration, sterile aqueous solutions of water-soluble salts (e.g., NaCl) can be employed. Additional or alternative carriers may include sesame or peanut oil, as well as aqueous propylene glycol. Aqueous solutions may be suitably buffered, if necessary, and the liquid diluent can first be rendered isotonic with sufficient saline or glucose. These aqueous solutions are especially suitable for intravenous, intramuscular, subcutaneous, intraperitoneal, and intratumoral (IT) injection. Intratumoral injection can be especially helpful for certain types of therapy, such as the treatment of cancer, including prostate cancer. Appropriate sterile aqueous media can be purchased (e.g., Sigma-Aldrich Corporation, St. Louis, Mo.) or can be prepared by standard techniques well known to those skilled in the art.

In some embodiments, the compositions are completely free of additives such as one or more of ethanol, dimethyl sulfoxide, or other organic solvents, phospholipids, castor oil, and castor oil derivatives. In other embodiments, the composition is substantially free of such components. As used herein, substantially free means that the composition contains less than about 2.5 wt. %, less than about 2 wt. %, less than about 1.5 wt. %, less than about 1 wt. %, less than about 0.5 wt. %, or less than about 0.25 wt. %. In some embodiments, certain additives can increase the stability of the micelles. In one embodiment, a surfactant can be included in the micelle (e.g., in about 0.25 wt. % to about 2.5 wt. %). For example, a suitable surfactant can be a negatively charged phospholipid, such as polyethylene glycol conjugated distearoyl phosphatidyl-ethanolamine (PEG-DSPE).

### Therapy Using Micelle Formulations

The micelles can be formulated into a pharmaceutical solution and administered to a patient. The pharmaceutical solution formulation can allow for delivery of a requisite amount of 17-AAG to the body within an acceptable time, for example, about 10 minutes, to about 3 hours, typically about 1 to about 2 hours, for example, about 90 minutes.

The administration can be parenteral, for example, by infusion, injection, or IV, and the patient can be a mammal, for example, a human. Upon administration, the micelles can circulate intact, dissociate into individual polymer chains, release active agents (either by diffusion or micelle dissociation), distribute into tissue (e.g. tumors), and/or undergo renal clearance. The schedule of these events cannot be predicted with specificity, and these events significantly influence the anti-tumor activity of the active agents, such as 17-AAG. In some embodiments, the drug-loaded micelles can extravasate into tumor interstitial. The active agent-containing micelles can hydrolyze to release the 17-AAG, which can then release the active agent from the micelle. The active agent can then diffuse into tumor cells. Another aspect of the invention includes the micelles crossing leaky vasculature and endocytosing into tumor cells, and inhibiting the tumor cell growth, and/or killing the tumor cells.

A disease, disorder, or condition can be treated by administering a pharmaceutical formulation of micelles that contain 17-AAG. Administration of the compositions described herein can result in a reduction in the size and/or the number of cancerous growths in a patient, and/or a reduction in one or more corresponding associated symptoms. When administered in an effective amount by the methods described herein, the compositions of the invention can produce a pathologically relevant response, such as inhibition of cancer cell proliferation, reduction in the size of a cancer or tumor, prevention of further metastasis, inhibition of tumor angiogenesis, and/or death of cancerous cells. The method of treating such diseases and conditions described below includes administering a therapeutically effective amount of a composition of the invention to a patient. The method may be repeated as necessary, for example, daily, weekly, or monthly, or multiples thereof.

Conditions that can be treated include, but are not limited to, hyperproliferative diseases, including cancers of the head and neck, which include tumors of the head, neck, nasal cavity, paranasal sinuses, nasopharynx, oral cavity, oropharynx, larynx, hypopharynx, salivary glands, and paragangliomas; cancers of the liver and biliary tree, particularly hepatocellular carcinoma; intestinal cancers, particularly colorectal cancer; ovarian cancer; small cell and non-small cell lung cancer; prostate cancer; pancreatic cancer; breast cancer sarcomas, such as fibrosarcoma, malignant fibrous histiocytoma, embryonal rhabdomysocarcoma, leiomysosarcoma, neurofibrosarcoma, osteosarcoma, synovial sarcoma, liposarcoma, and alveolar soft part sarcoma; neoplasms of the central nervous systems, particularly brain cancer; and/or lymphomas such as Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, or T-cell anaplastic large cell lymphoma.

Non-cancer conditions that are characterized by cellular hyperproliferation can also be treated using the methods described herein. For example, 17-AAG can be administered according to the methods described herein to treat conditions that are characterized by cellular hyperproliferation. Illustrative examples of such non-cancer conditions, disorders, or diseases include, but are not limited to, atrophic gastritis, inflammatory hemolytic anemia, graft rejection, inflammatory neutropenia, bullous pemphigoid, coeliac disease, demyelinating neuropathies, dermatomyositis, inflammatory bowel disease (ulcerative colitis and/or Crohn's disease), multiple sclerosis, myocarditis, myositis, nasal polyps, chronic sinusitis, pemphigus vulgaris, primary glomerulonephritis, psoriasis, surgical adhesions, stenosis or restenosis, scleritis, scleroderma, eczema (including atopic dermatitis, irritant dermatitis, allergic dermatitis), periodontal disease (i.e., periodontitis), polycystic kidney disease, and type I diabetes. Other examples include vasculitis, e.g., Giant cell arteritis (temporal arteritis, Takayasu's arteritis), polyarteritis nodosa, allergic angiitis and granulomatosis (Churg-Strauss disease), polyangitis overlap syndrome, hypersensitivity vasculitis (Henoch-Schonlein purpura), serum sickness, drug-induced vasculitis, infectious vasculitis, neoplastic vasculitis, vasculitis associated with connective tissue disorders, vasculitis associated with congenital deficiencies of the complement system, Wegener's granulomatosis, Kawasaki's disease, vasculitis of the central nervous system, Buerger's disease and systemic sclerosis; gastrointestinal tract diseases, e.g., pancreatitis, Crohn's disease, ulcerative colitis, ulcerative proctitis, primary sclerosing cholangitis, benign strictures of any cause including ideopathic (e.g., strictures of bile ducts, esophagus, duodenum, small bowel or colon); respiratory tract diseases (e.g., asthma, hypersensitivity pneumonitis, asbestosis, silicosis and other forms of pneumoconiosis, chronic bronchitis and chronic obstructive airway disease); nasolacrimal duct diseases (e.g., strictures of all causes including ideopathic); eustachean tube diseases (e.g., strictures of all causes including idiopathic); as well as neurological diseases, fungal diseases, viral infections, and/or malaria.

The terms “treat” and “treatment” refer to any process, action, application, therapy, or the like, wherein a mammal, including a human being, is subject to medical aid with the object of improving the mammal's condition, directly or indirectly. Treatment typically refers to the administration of an effective amount of a micelle composition as described herein.

The terms “effective amount” or “therapeutically effective amount” are intended to qualify the amount of a therapeutic agent required to relieve to some extent one or more of the symptoms of a condition, disease or disorder, including, but not limited to: 1) reduction in the number of cancer cells; 2) reduction in tumor size; 3) inhibition of (i.e., slowing to some extent, preferably stopping) cancer cell infiltration into peripheral organs; 3) inhibition of (i.e., slowing to some extent, preferably stopping) tumor metastasis; 4) inhibition, to some extent, of tumor growth; 5) relieving or reducing to some extent one or more of the symptoms associated with the disorder; and/or 6) relieving or reducing the side effects associated with the administration of active agents.

The term “inhibition,” in the context of neoplasia, tumor growth or tumor cell growth, may be assessed by delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition, can be referred to as prevention or chemoprevention. The inhibition can be about 10%, about 25%, about 50%, about 75%, or about 90% inhibition, with respect to progression that would occur in the absence of treatment.

Using a pharmaceutical solution formulation of this invention, active agents such as 17-AAG and/or an anticancer or cytotoxic agent may be administered in a dose ranging from about 4 mg/m² to about 4000 mg/m², depending on the frequency of administration. In one embodiment, a dosage regimen for 17-AAG can be about 400-500 mg/m² weekly, or about 450 mg/m² weekly. See Banerji et al., Proc. Am. Soc. Clin. Oncol., 22, 199 (2003, abstract 797). Alternatively, a dose of about 300 mg/m² to about 325 mg/m², or about 308 mg/m² weekly can be administered to the patient. See Goetz et al., Eur. J. Cancer, 38 (Supp. 7), S54-S55 (2002). Another dosage regimen includes twice weekly injections, with doses ranging from about 200 mg/m² to about 360 mg/m² (for example, about 200 mg/m², about 220 mg/m², about 240 mg/m², about 250 mg/m², about 260 mg/m², about 280 mg/m², about 300 mg/m², about 325 mg/m², 340 mg/m², about 350 mg/m², or about 360 mg/m², depending on the severity of the condition and health of the patient). A dosage regimen that can be used for combination treatments with another drug, such as docetaxel, can be to administer the two drugs every three weeks, with the dose of 17-AAG of about 500 mg/m² to about 700 mg/m², or up to about 650 mg/m² at each administration.

### Drug Combinations in PEG-PLA Micelles

In addition to 17-AAG, other active agents can be encapsulated in the micelles described herein by the same preparatory procedures. For example, paclitaxel-containing micelles can be used in a formulation along with 17-AAG-containing micelles (a “simply mixed” micelle formulation, wherein each micelle contains a different active agent). Thus, the invention provides a composition that includes both 17-AAG and a second active agent, wherein both the 17-AAG and the second active are solubilized in an aqueous solution by encapsulation within PEG-PLA micelles. For example, the composition can include both paclitaxel and 17-AAG in the micelles of a simply mixed micelle formulation. Formulations can also be prepared where both 17-AAG and a second active agent are dissolved in the micelle preparation procedure, forming individual micelles that contain both 17-AAG and the second active agent, thus providing “physically mixed” micelles, wherein one or more of the micelle contains two different active agent.

Accordingly, 17-AAG can be administered (e.g., in micelles as described herein) in combination with other active agents (e.g., anti-cancer or cytotoxic agents), including alkylating agents, angiogenesis inhibitors, antimetabolites, DNA cleavers, DNA crosslinkers, DNA intercalators, DNA minor groove binders, heat shock protein 90 (Hsp 90) inhibitors, histone deacetylase inhibitors, microtubule stabilizers, nucleoside (purine or pyrimidine) analogs, proteasome inhibitors, topoisomerase (I or II) inhibitors, tyrosine kinase inhibitors. Specific active agents include β-lapachone, 17-DMAG, bicalutamide, bleomycin, bortezomib, busulfan, calicheamycin, callistatin A, camptothecin, capecitabine, carzelesin, CC-1065, cisplatin, clanfenur, cryptophycins, cyclosporine A, daunorubicin, diazepam, discodermolide, docetaxel, doxorubicin, duocarmycin, dynemycin A, epothilones, etoposide, floxuridine, fludarabine, fluorouracil, gefitinib, geldanamycin, gemcitabine, hydroxyurea, imatinib, interferons, interleukins, itraconazole, irinotecan, leptomycin B, methotrexate, mitomycin C, oxaliplatin, paclitaxel, spongistatins, suberoylanilide hydroxamic acid (SAHA), teniposide, thiotepa, topotecan, trichostatin A, vinblastine, vincristine, and vindesine. The co-administered anti-cancer or cytotoxic agent can also be a protein kinase inhibitor. Examples of protein kinase inhibitors include rapamycin; quinazolines, particularly 4-anilinoquinazolines such as Iressa (AstraZeneca; N-(3-chloro-4-fluorophenyl)-7-methoxy-6-[3-(4-morpholinyl)propoxy]-4-quinazolinamine) or Tarceva (Roche/Genentech; N-(3-ethynylphenyl)-6,7-bis(2-m-ethoxyethoxy)-4-quinazolinamine monohydrochloride); phenylamino-pyrimidines such as Gleevec (Novartis; 4-[(4-methyl-1-piperazinyl)methyl]-N-[4-methyl-3-[[4-(3-pyridinyl)-2-pyrimidinyl]amino]phenyl]-benzamide); pyrrolo- or pyrazolopyrimidines such as BIBX 1382 (Boehringer Ingelheim; N8-(3-chloro-4-fluorophenyl)-N-2-(1-methyl-4-piperidinyl)-pyrimido[5,4-d]pyrimidine-2,8-diamine); indoles or oxindoles such as Semaxinib (Pharmacia; 3-[(3,5-dimethyl-1H-pyrrol-2-yl)methylene]-1,3-dihydro-2H-indol-2-one); benzylidene malononitriles; flavones such as flavopiridol (Aventis; 2-(2-chlorophenyl)-5,7-dihydroxy-8-[(3S,4R)-3-hydroxy-1-methyl-4-piperidinyl]-4H-1-benzopyran-4-one); staurosporines such as CEP-701 (Cephalon); antibodies such as Herceptin (Genentech); and/or ribozymes such as Angiozyme (Ribozyme Pharmaceuticals).

The combination of 17-AAG and certain other active agents has synergistic anticancer activity. For example, synergy can be observed when 17-AAG is administered in combination with paclitaxel, docetaxel, etoposide, as well as several other agents listed above. In various embodiments, these two agents can be combined in the same micelles (physically mixed formulations), or micelles individually incorporating paclitaxel and 17-AAG in separate micelles can be combined into one treatment formulation (a simply mixed formulation) for administration to a patient. In other embodiments, the 17-AAG micelles can be administered before, concurrently, or after administration of a drug other than 17-AAG. The drug other than 17-AAG can be administered in by any effective means, including administration by micelle encapsulation as described herein. The 17-AAG can sensitize the patient so that lower amounts of the other drug are necessary for effective treatment.

Additionally, it was unexpectedly found that the dual-agent micelles could be prepared such that the drug loading was within about 20% of the maximum loading that was obtainable for single-agent micelles. In addition to this surprising result, it was found that PEG-PLA micelles that contain both active agents in their cores are more stable with respect to the loss of one of the actives. Accordingly, it was discovered that in micelles containing two active agents, the actives can interact in such a manner as to increase the stability of the micelle, with respect to release of the actives. Thus, micelles that contain 17-AAG and a second active agent, such as paclitaxel, docetaxel, or etoposide, have been found to be more stable than micelles that incorporate only one of the active agents.

These discoveries allow for the administration of an Hsp 90 inhibitor (e.g., 17-AAG) and a chemotherapeutic agent (e.g., paclitaxel, docetaxel, or etoposide, among others) without resorting to the use of significant amounts of organic solvents or surfactants in the treatment formulations. In clinical trials, the combination of 17-AAG and paclitaxel requires DMSO and Cremophor® EL, a four component cocktail. The components of such formulations have been found cause significant adverse side-effects in some patients. Also, the two drugs cannot be mixed and infused together, and any drug synergy is achieved by concurrent drug administration (Solit et al., Cancer Res., 2003; 63:2139-2144). It is known that paclitaxel/PEG-PLA can be safely administered to patients. For example, Genexol-PM is currently in phase II clinical trials. Also, 17-AAG can be co-loaded into PEG-PLA micelles without requiring a significant increase in the number of the micelles. Such formulations can also avoid the use of organic solvents or other surfactants.

Accordingly, various conditions can be treated using the amphiphilic block copolymer (ABC) micelle systems described herein. Drug synergy can be achieved by use of the micelles, which can reduce the toxicity of a treatment regimen due to drug encapsulation within the micelle delivery vehicles. Combinations of active agents can be used in the micelles. Simply mixed and physically mixed formulations allow for the administration of two different active agents with one administration, e.g., an IV infusion. Certain useful combinations and techniques are described in U.S. Pat. No. 7,221,562 (Rosen et al.). Other amphiphilic copolymers that may be used in embodiments of the invention include those described in U.S. Pat. No. 4,745,160 (Churchill et al.).

The micelle compositions disclosed herein provide for improved formulation that have unexpectedly high loading capacity for 17-AAG and can be used to prepare controlled release formulations. It was also discovered that the drug loading dual-active micelles can approach, or be equal to, the drug loading capacity of single agent micelles. Additionally, interaction between the actives in the dual-active micelles can increase the stability of such micelles. For example, 17-AAG can act as a stabilizer for dual agent micelle formulations, with respect to both simply mixed formulations and also physically mixed formulations.

The following Examples are intended to illustrate the above invention and should not be construed as to narrow its scope. One skilled in the art will readily recognize that the Examples suggest many other ways in which the present invention could be practiced. It should be understood that many variations and modifications may be made while remaining within the scope of the invention.

### EXAMPLES

### Example 1

### 17-AAG Encapsulation in PEG-PLA Micelles

Preparation of 17-(allylamino)-17-demethoxygeldanamycin. (17-AAG) was synthesized in the lab from geldanamycin (GA) (LC Laboratories, Woburn, Mass.). Briefly, 100 mg of GA (0.2 mmol) was dissolved in 2 mL of dry CH₂Cl₂. Next, 5 equivalents of allylamine (57.1 g/mol, d=0.763 g/mL) was added dropwise to the flask. The reaction was stirred at room temperature (RT; ˜23° C.) under low light until complete by TLC analysis (approx. 2 days) (95:5 CHCl₃:MeOH, Rt. 0.21), precipitated with hexane (3×), centrifuged at 2000 g's for 15 minutes, and evaporated to dryness. Yield: 95 mg, 95%; MS m/z 584 (M); ¹H NMR (CDCl₃) δ 0.99 (m, 6H, 10-Me, 14-Me), 1.25 (t, 1H, H-13), 1.60-1.85 (br m, 6H, H-13, H-14, 8-Me), 2.05 (s, 3H, 2-Me), 2.46 (br m, 2H, H-15), 2.83-2.90 (br m, 3H, H-10), 3.27 (s, 3H, OMe), 3.36 (s, 3H, OMe), 3.40 (t, 1H, H-12), 3.58-3.68 (br m, 2H, H-11, H-23), 4.31 (d, 1H, H-7), 5.10 (br s, 1H), 5.21-5.55 (br m, 3H, H-9, H-24), 5.86-5.99 (br t, 2H, H-5, H-23), 6.59 (t, 1H, H-4), 6.94 (d, 1H, H-3), 7.28 (br s, 1H, H-19).

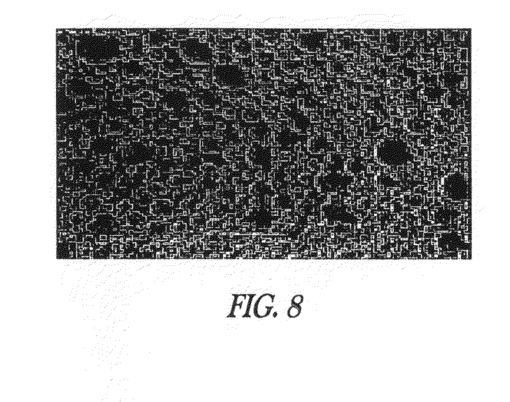

Preparation and characterization of drug loaded PEG-PLA micelles. 17-AAG was formulated by dissolving it with PEG-PLA (12:6 kDa) (Polymer Source, Montreal, Canada) in dimethylacetamide (DMAc) and dialyzing against H₂O, following procedures by Kataoka and coworkers (J. Control. Release 62(1-2) (1999) 89-100). For example, 5 mg of 17-AAG and 45 mg of PEG-PLA (10:90 w/w) were dissolved in 10 mL DMAc. The resulting solution was dialyzed against H₂O in 3500 MWCO tubing (SpectraPor). Resulting micelles were centrifuged at 5000 g's for 10 min to precipitate unincorporated drug. Incorporation into micelles was verified using aqueous GPC (Shodex SB-806M) by confirming equivalent retention times based on refractive index for the micelles and absorbance of 17-AAG (UV λ332). Micelle solutions were concentrated by rotary evaporation at reduced pressure at room temperature, followed by centrification (5000 g's for 10 minutes). FIG. 8 shows a transmission electron microscopy (TEM) image of isolated PEG-PLA micelles (18,000×).

Quantitative drug loading in micelles was determined by monitoring the area under the curve (AUC) for 17-AAG (based on a 17-AAG calibration curve) through reverse-phase HPLC (Shodex C18 column, 65-82.5: 35-17.5 MeOH to 55% MeOH+0.2% formic acid gradient, 40° C., 332 nm detection). Effective diameters of PEG-PLA micelles, with and without drugs, were measured using a Brookhaven dynamic light scattering apparatus (100 mW, 532 nm laser) with Gaussian intensity fitting. The critical micelle concentration (CMC) for these PEO-b-PDLLA micelles was determined by measuring the 339/334-nm excitation ratio of pyrene in the presence of various concentrations of PEG-PLA (3×10⁻⁵ mg·mL⁻¹ to 1 mg·mL⁻¹).

Briefly, PEO-b-PDLLA micelles were prepared as described above in serial dilutions and incubated with 0.6 μM pyrene for 1 hour at 80° C., allowed to sit in the dark for 15 hours at RT, and the fluorescence emission of pyrene was measured at 390 nm (RF-5301 PC spectrofluorophotometer, Shimadzu). Pyrene undergoes well-known photophysical changes in response to its microenvironment polarity (Colloids Surf., A Physiochem. Eng. Asp. 118 (1996) 1-39). A sharp increase in the ratio of 339/334 nm excitation occurs at the CMC as the pyrene preferentially partitions into the hydrophobic cores of PEO-b-PDLLA micelles (J. Control. Release 77(1-2) (2001) 155-160).

### Example 2

### A Cremophor®-Free Formulation for 17-AAG using PEG-PLA Micelles: Characterization and Pharmacokinetics in Rats

The preparation of amphiphilic block copolymer (ABC) micelles such as degradable amphiphilic diblock polymers composed of poly(ethylene oxide)-block-poly(D,L-lactide) (PEG-PLA) as nanocarriers for solubilizing 17-AAG was described above in Example 1. The pharmacokinetic behavior of these nanocarriers is compared with a current formulation of 17-AAG in CrEL-EtOH-PEG400 in this Example.

PEG-PLA micelle drug release studies. Release experiments were based on the methodology of Eisenberg and coworkers (Soo et al., Langmuir 18 (2002) 9996-10004), with previously reported modifications for temperature and pH control (Forrest et al., J. Control. Release 116(2) (2006) 139-149). Micelle drug solutions were prepared at 0.3 mM PEG-PLA polymer with 10% w/w drug as described above, and 2.0 mL of the micelle solution was injected into 10,000 MWCO dialysis cassettes (Pierce, Rockford, Ill.) (n=3). Dialysis cassettes were placed in a well-mixed temperature-controlled water bath at 37° C., with bath volume refreshment every 15 to 20 minutes. Peristaltic pumps under computer control separately injected 50 g/L solutions of dibasic and monobasic phosphate to maintain pH at 7.4±0.1 (apparatus built in-house). At fixed time points, dialysis cassette volumes were made up with ddH₂₀ to 2 mL, if necessary, and 100 μL aliquots were withdrawn. This was mixed with 100 μL MeOH and 40 μL of the mixture was analyzed by reverse-phase HPLC (Shodex C18 column; 65-82.5: 35-17.5 of A to B where A: MeOH and B: 55% MeOH+0.2% formic acid; 40° C.; 332 nm detection).

In vitro cytotoxicity studies. PC-3 human prostate cells (ATCC CRL-1435) were grown in RPMI 1640 (Hyclone, Logan, Utah) and MCF-7 human breast cancer cells (ATCC HTB-22) were cultured in DMEM (Hyclone), both supplemented with 10% Fetal Bovine Serum (Hyclone), 100 μg/mL penicillin-streptomycin (Cambrex Biosciences, Baltimore, Md.), and 2 mM L-glutamine (Cambrex Biosciences). Cell lines were plated in 96-well plates at an initial density of 3000 cells per well in 90 μL of appropriate media, and maintained at 37° C. under a 5% CO₂ atmosphere. After 24 hours, 17-AAG in DMSO was diluted 10-fold with growth media and added to wells (3 wells in duplicate, n=6) as 10-μL aliquots (1% v/v final DMSO concentration). Drug-loaded micelles or micelles alone were formulated in MilliQ water (following dialysis of DMSO to form the micelles). It was not necessary to dilute the samples further before addition to wells. Cells were incubated with all the test compounds for 72 hours. Following, the metabolic rate of cells was determined by using resazurin dye (Sigma-Aldrich). The concentrations inhibiting cell growth by 50% (IC₅₀) relative to controls were determined by fitting the data to a Hill-Slope curve (Sigma Plot 9.0, Systat Software, Inc.) and is reported as the average of separate measurements±the standard deviation.

Pharmacokinetic studies. The standard vehicle for 17-AAG was formulated according to Zhong et al. (U.S. application Publication No. 2005/0256097), where 15 mg/mL 17-AAG in 2:1:1 EtOH:CrEL:PEG400 was diluted to 3 mg/mL immediately before injection. The methods for incorporation and drug release for these micelles were performed as described with slight modifications (Forrest et al., J. Control. Release 110(2) (2006) 370-377). Male Sprague Dawley rats were cannulated via the right jugular vein and dosed intravenously with either the new formulation (1.5 mg/mL 17-AAG in PEG-PLA) or the standard formulation of CrEL-EtOH-PEG400, each at 10 mg/kg (n=5 for each treatment group). After dosing, serial blood samples (˜0.30 mL) and urine samples were collected up to 24 hours.

To 100 μL of serum or urine, 100 μL of internal standard was added (25 μg/mL geldanamycin). The samples were extracted with 1 mL of EtOAc and the organic fractions were dried and concentrated. The residue was reconstituted in 400 μL of the initial mobile phase, centrifuged, and 150 μL was injected into an RP-HPLC. The 17-AAG was analyzed at 332 nm with an internal standard at 305 nm on a Genesis 3 μm C18 33 mm×4.6 mm column at 1 mL/min in A: 50 mM acetic acid+10 mM TEA and B: MeOH+10 mM TEA (0-3 min 40% B, 3.01-11 min 80% B, 11.01-18 min 40% B). Pharmacokinetic parameters were calculated using WinNonlin® software (ver. 5.01) and non-compartmental modeling. All animal studies were conducted in accordance with “Principles of laboratory animal care” (NIH publication No. 85-23, revised 1985).

Biodistribution studies. To assess the effect of the formulations on the tissue distribution of 17-AAG, rats (n=5 for each group, 200-240 g) were cannulated and intravenously administered with 17-AAG in CrEL-EtOH-PEG400 or 17-AAG in PEG-PLA micelles, all equivalent to the previous pharmacokinetic studies. At 3 hours after formulation injection, each animal (n=5 for each time point) was anaesthetized and exsanguinated by cardiac puncture. Brain, lungs, heart, liver, spleen, kidneys, urinary bladder, muscle, and bone, as well as samples of whole blood and serum, were collected. Tissue samples were blotted with paper towels, washed in ice-cold saline, bottled to remove excess fluid, weighed and rapidly frozen in liquid nitrogen, pulverized to a fine powder with a mortar and pestle under liquid nitrogen, and stored at −70° C. until assessed for drug concentrations by HPLC analysis.

Data Analysis. Compiled data are presented as mean and standard error of the mean (mean±SEM) or mean and standard deviation (mean±SD) where otherwise indicated. Where possible, the data were analyzed for statistical significance using NCSS Statistical and Power Analysis software (NCSS, Kaysville, Utah). Student's t-test was employed for unpaired samples with a value of p<0.05 being considered statistically significant.

### Results and Discussion

Geldanamycin (GA) is poorly water soluble. GA formulations in typical pharmaceutical carriers are severely hepatotoxic because the liver is the primary elimination route. Analogues, such as 17-DMAG and 17-AAG exhibit lower hepatotoxicity than GA, but may be characterized by increased nonspecific toxicities due in part to their wide distributions. Accordingly, a nanocarrier formulation is highly desired. These experiments sought to determine if the biocompatibility, ease of manufacture, kinetic and thermodynamic stability, and the ability of PEG-PLA micelles to solubilize large quantities of lipophilic drugs could provide a nanocarrier system that could be employed in conjunction with the micelle formulations of the invention. Various quantities of 17-AAG were solubilized in PEG-PLA micelles (Table 1).

TABLE 1
Summary of micelle loading.

| Properties | 0.3 mM PEG-PLA solution |
|---|---|
| Initial 17-AAG:¹ | 0.45 mg/mL |
| 17-AAG solubilized: | 0.083 ± 0.012 mg/mL |
| After concentration:² | 1.5 ± 0.2 mg/mL |
| 17-AAG loaded:³ | 0.024 ± 0.003 mg/mg |
| mol 17-AAG:mol PEG-PLA: | 0.73 ± 0.09 |
| Efficiency:⁴ | 19 ± 3% loaded |

¹Solutions were prepared with 11.4% w/w 17-AAG based on PEG-PLA content.
²Micelles concentrated by rotary evaporation.
³Loading based on weight 17-AAG incorporated versus total weight of carrier, including 17-AAG and PEG-PLA.
⁴Efficiency based on total drug solubilized versus initial drug concentration.

The enhanced solubility and lack of harsh surfactants for delivering 17-AAG demonstrates that this micelle formulation is far superior to present delivery vehicles that contain CrEL.

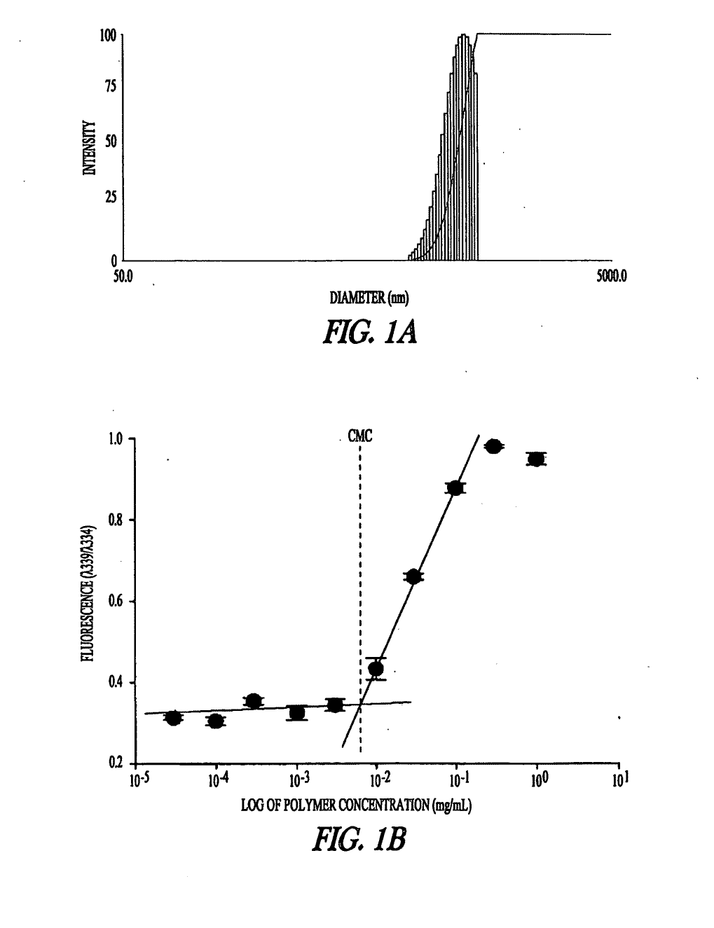

Micelle physical characterization. Dynamic light scattering (Brookhaven Instruments Corporation) revealed PEG-PLA (12:6 kDa) micelles with average sizes of 242±5 nm without drugs, and 257±2 nm loaded with drugs, solubilizing up to 1.5 mg/mL of 17-AAG. It was found that PEG-PLA (12:6 kDa) had low critical micelle concentrations (CMC) of 350 nM (FIG. 1(b)), indicating that the micelles had high thermodynamic stability as given by the Gibb's free energy of micellization:

ΔG=RT In(CMC).

The size and CMC reported here for PEG-PLA (12:6 kDa) micelles are considerably larger than previously reported by Kataoka et al. (J. Control. Release 62(1-2) (1999) 89-100). Encapsulation 17-AAG in micelles was carried out using DMSO as the organic solvent, but it was found that use of DMAc was advantageous when forming micelles with dialysis methodology. The loading experiments were investigated at 10% w/w to evaluate the suitability of PEG-PLA micelles for solubilizing 17-AAG.

The reported values of 17-AAG loading in Table 1 represent 10% w/w loading of 17-AAG based on PEG-PLA. Drug incorporation into micelles was verified using aqueous GPC (Shodex SB-806M) by monitoring equivalent retention times in refractive index (micelles) and 17-AAG (UV λ332). The mole ratio of 17-AAG to PEG-PLA ranged from 0.73±0.09 to 1, with a 17-AAG loading efficiency of 19±3% w/w. After concentration by rotary evaporation, the maximum solubility of 17-AAG in 0.3-mM PEO₆₀₀₀-b-PDLLA₁₂₀₀₀ was about 1.5±0.2 mg/mL, an improvement of about 150-fold over 17-AAG (˜10 μg/mL according to the National Cancer Institute). Incorporating a 1:1 ratio of alpha-tocopherol to drug was attempted to improve loading as previously reported for rapamycin (J. Control. Release 110(2) (2006) 370-377), but the resulting micelles were large (>500 nm) and were unstable. The drug precipitated out of solution after 4-5 hours even at 4° C. It was also found that drug loading in PEG-PLA (12:12 kDa) micelles was very poor (<0.5 mg/mL) compared to PEG-PLA (12:6 kDa) micelles (1.5 mg/mL), also with and without alpha-tocopherol.

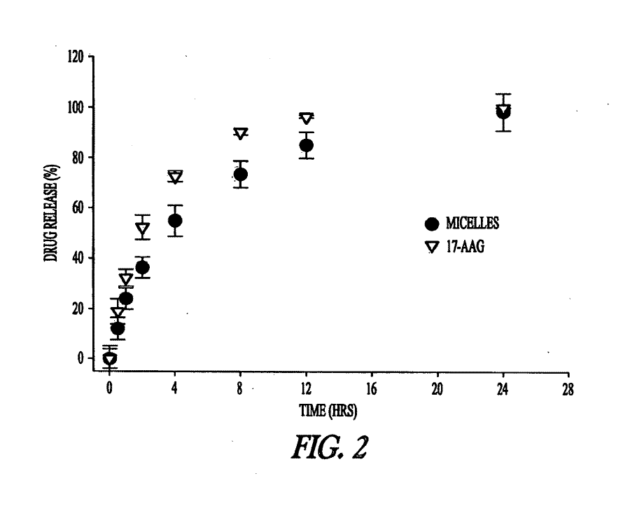

17-AAG release studies. The in vitro release kinetics of 17-AAG from PEG-PLA micelles at nominal body temperature were investigated by dialysis of drug-loaded micelles against 37° C. water. Due to the low solubility of 17-AAG, the release medium would have saturated quickly without continuous purging of the bath. Therefore, a constraint in the analysis of the release data was the inability to measure the release of 17-AAG directly from PEG-PLA micelles. Once the drug released from PEG-PLA micelles, it had to diffuse across the dialysis membrane, introducing a second diffusion barrier and complicating the analysis. To determine if the dialysis membrane contributed significantly to the overall release rate, cassettes were loaded with free 17-AAG (initially dissolved in minimal amount of MeOH before being diluted with water) and the release kinetics were measured. The PEG-PLA micelles demonstrated release with a half-life of ca. 4 hours (FIG. 2), a 2-fold improvement over that of the drug alone.

In vitro cytotoxicity studies. Growth inhibitory activity of the micelle-encapsulated 17-AAG against MCF-7 human breast cancer and PC-3 prostate cancer cells were determined in vitro using resazurin dye to assay for metabolic activity. 17-AAG was active at sub-micromolar concentrations with IC₅₀ of 22±14 nM in MCF-7 and IC₅₀ 74±14 nM in PC-3 cells; all numbers within the range reported by the National Cancer Institute: IC₅₀ of 12.6 nM for MCF-7 and 5Q.1 nM for PC-3, respectively. In comparison, the drug-encapsulated micelles had IC₅₀ of 160±56 nM in MCF-7 and 536±13 nM in PC-3 cells (Table 2).

TABLE 2
Cytotoxicity summary for 17-AAG, 17-AAG drug-loaded
micelles, and micelles alone.

| | MCF-7 | PC-3 |
|---|---|---|
| Test agent | IC₅₀ (nm)^{a} | IC₅₀ (nm)^{a} |
| 17-AAG | 22 ± 14 | 74 ± 14 |
| PEG-PLA drug-loaded micelles | 160 ± 57^{b} | 536 ± 13^{b} |
| PEG-PLA micelles | >10 000 | >10 000 |

^{a}Cells were incubated with test agents for 72 hours (n = 6), and growth inhibition was measured by monitoring metabolic rates using resazurin dye.
^{b}Denotes statistically significant differences (p < 0.05) between 17-AAG alone and 17-AAG in PEG-PLA micelles.

This difference in IC₅₀ values may be due to prolonged release of the drugs from micelles, as well as the eventual equilibrium reached between release and drug partitioning back into the micellar core in a closed system. Micelles alone showed no apparent toxicity in the cell lines investigated (IC₅₀>10,000 nM). Although the encapsulated drugs appear less active than 17-AAG, nanoencapsulation is believed to increase the overall efficacy of 17-AAG delivery in vivo due to enhanced tumor accumulation. Similarly, Li et al. reported a poly-(L-glutamic acid)-paclitaxel conjugate was significantly less potent than paclitaxel in vitro but demonstrated superior activity in vivo due to enhanced accumulation in tumors (Cancer Res. 58 (1998) 2404-2409).

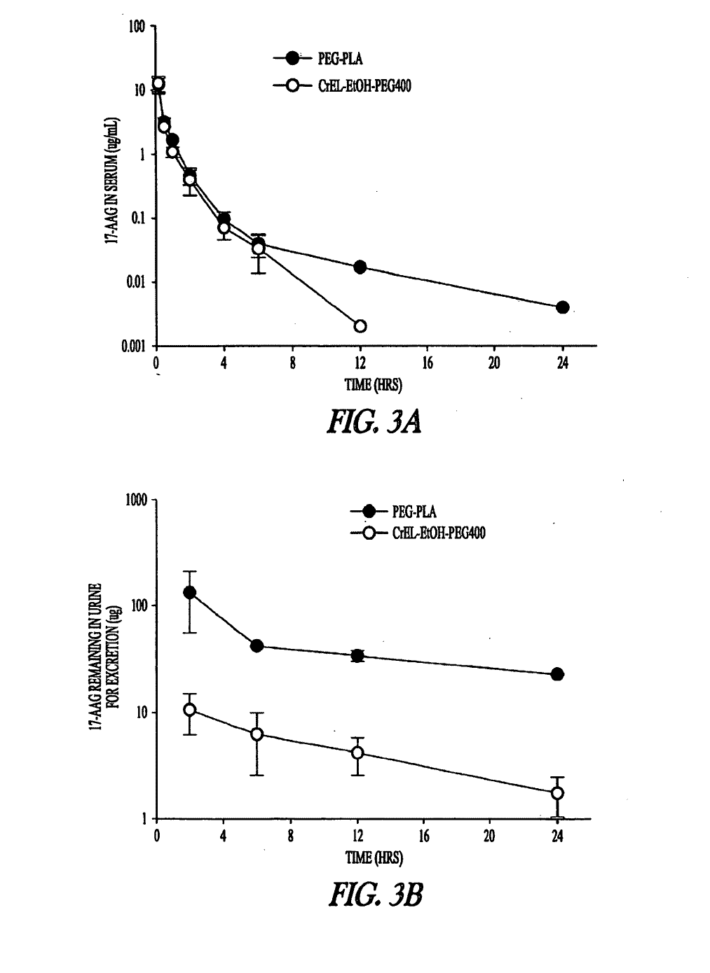

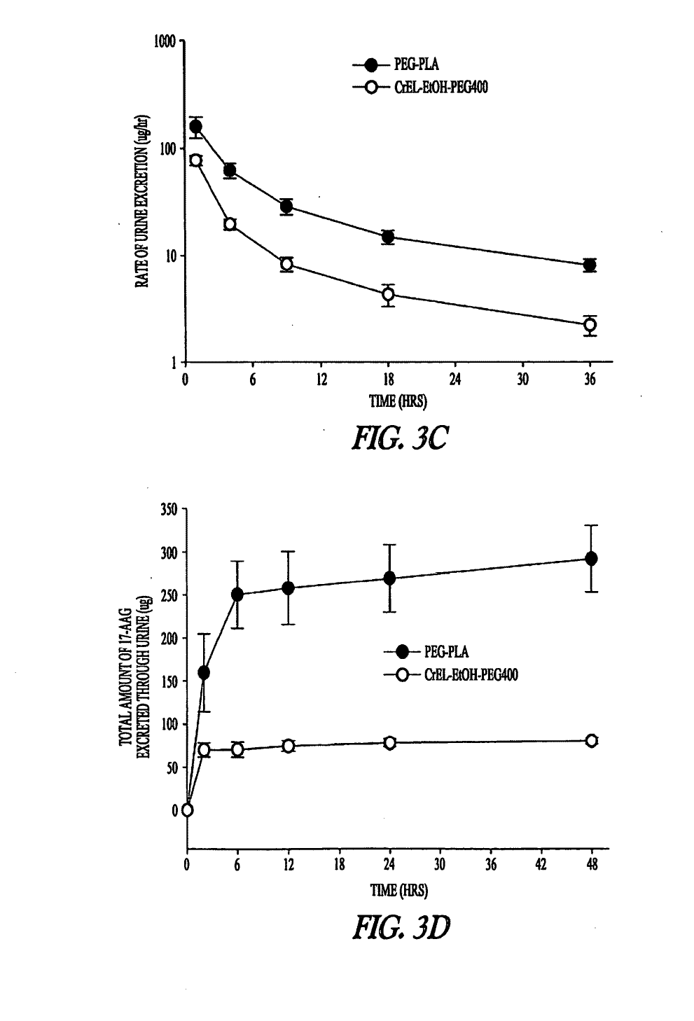

Pharmacokinetic studies. Differences in the pharmacokinetic profile of PEG-PLA (12:6 kDa) micelle formulation compared to the standard formulation with CrEL-EtOH-PEG400 are illustrated in FIG. 3. The micellar formulation demonstrated increased serum AUC 24-hrs after injection (FIG. 3a) as observed by an effective quantification of 17-AAG from the micellar formulation up to 24 hours compared to the control formulation (effective quantification only up to 12 hours before going below the limit of quantification). The micellar formulation also exhibited increased presence of 17-AAG in the urine after 24 hours (FIG. 3b), greater rate of renal clearance over 36 horrs (FIG. 3c), and higher levels of drug in the urine 48 hours post-injection (FIG. 3d). The serum area under the curve (AUC) of PEG-PLA micelles was 1.3-fold that of the standard formulation. At 10 mg/kg (Table 3), the total clearance (CL total) decreased 1.3 fold with the micelle formulation as compared to the standard vehicle, the renal clearance of the drug (CL renal) increased (4.3 fold) with the micelle formulation, as compared to the standard vehicle which demonstrated a higher (1.5 fold) hepatic clearance (CL hepatic).

TABLE 3
Pharmacokinetic parameters of 17-AAG in CrEL-EtOH-PEG400
and 17-AAG in PEO-b-PDLA micelles in rats.

| | PEG-PLA | | CrEL-EtOH-PEG400 | |
|---|---|---|---|---|
| Parameter | 10 mg/kg | | 10 mg/kg | |
| Mean (SEM) | (n = 5) | | (n = 5) | |
| AUC_{0→ ∞}(μg h mL⁻¹) | 6.42 | (1.42) | 4.85 | (1.06) |
| V_{d} (L kg⁻¹) | 8.9 | (2.3)^{a} | 5.2 | (2.2) |
| CLₜₒₜₐₗ (L h⁻¹ kg⁻¹) | 1.64 | (0.36)^{a} | 2.17 | (0.047) |
| t_{1/2} serum (h) | 4.21 | (1.92)^{a} | 1.58 | (0.38) |
| K_{E} (h⁻¹) | 0.21 | (0.095)^{a} | 0.47 | (0.11) |
| MRT (h) | 0.90 | (0.16) | 0.93 | (0.19) |
| t_{1/2} urine (h) | 13.4 | (4.44) | 10.9 | (0.73) |
| K_{E} urine (h⁻¹) | 0.058 | (0.019)^{a} | 0.064 | (0.0043) |
| Fe (%) | 11.7 | (1.55)^{a} | 1.66 | (1.55) |
| CL renal (L h⁻¹ kg⁻¹) | 0.18 | (0.02)^{a} | 0.043 | (0.041) |
| CL hepatic (L h⁻¹ kg⁻¹) | 1.45 | (0.34)^{a} | 2.12 | (0.43) |
| Extraction Ratio (ER) | 0.083 | (0.020)^{a} | 0.12 | (0.025) |
| Estimated | 91.7 | (1.98) | 87.8 | (2.48) |
| bioavailability (%) | | | | |

^{a}Denotes statistically significant differences (p < 0.05) between standard formulation with CrEL-EtOH-PEG400 and 17-AAG in PEG-PLA micelles.

The PEG-PLA (12:6 kDa) micelle formulation increased the serum half-life (t_{1/2}) of the drug 2.7-fold. An increase (1.7 fold) in the volume of distribution (V_{d}) with the micelle formulation was observed due to its and prolonged presence in the blood as observed by a lower total clearance (1.3-fold decrease), and higher half-life in serum (2.7-fold increase) and in urine (1.2-fold increase) compared to the control standard vehicle formulation. The renal clearance of the drug (CL renal) increased (4.3 fold) with the micelle formulation as compared to the standard vehicle, which demonstrated a higher (1.5 fold) hepatic clearance (CL hepatic). Finally, no significant difference was found in mean residence time (MRT) between the two formulations.

Although overall the pharmacokinetic parameters between the two formulations are not dramatically different, the results herein demonstrate that a drastic reduction in negative side effects can be provided by the 17-AAG PEG-PLA micelle nanocarrier formulation. The standard formulation includes CrEL, a harmful surfactant known to cause anaphylaxis in patients. Use of a CrEL formulation requires pretreatment with anti-histamines and steroids (Sydor et al., Proc. Nat. Acad. Sci. USA, 2006; 103(46), 17408-13). The micelle formulations according to the invention would not require such pretreatments.

The 17-AAG in PEG-PLA micelles was well tolerated in rats. No acute signs of toxicity were observed throughout the length of the study. Also, no mortality was observed with the nanocarrier formulation compared to a 35% mortality within 24 hours observed with the standard formulation of CrEL-EtOH-PEG400. Thus, the 17-AAG in PEG-PLA micelle nanocarrier formulation can retain the pharmacokinetic disposition of 17-AAG without the need for toxic agents such as EtOH and CrEL. The formulation thus provides a new method for using the promising chemotherapeutic agent 17-AAG in cancer therapy.

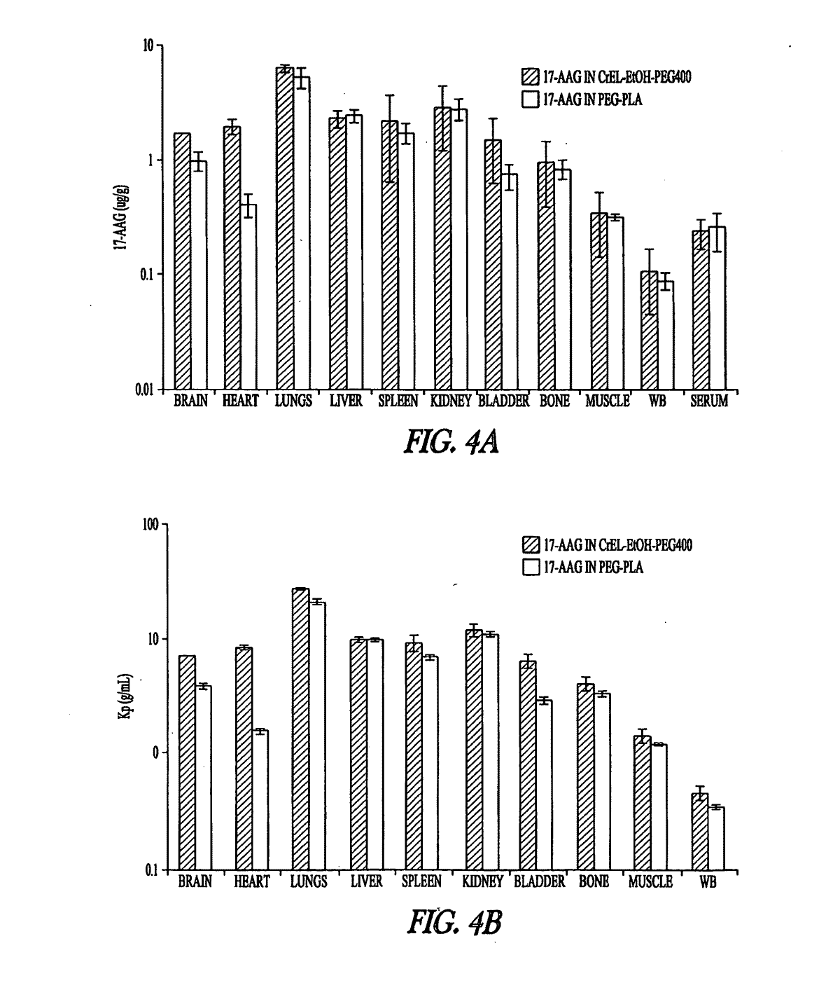

Biodistribution Studies. Quantifiable amounts of 17-AAG were observed in all assayed tissues (FIG. 4(a)). The tissue collection was performed at 3 hours post IV administration of the different formulations at 10 mg/kg. The order of 17-AAG concentrations from highest to lowest for the control formulation was: lungs>kidney>liver>spleen>heart>brain>urinary bladder>bone>muscle>serum>whole blood. For the micellar formulation, the order from highest concentration to lowest was: lungs>kidney>liver>spleen>brain>bone>urinary bladder>heart>muscle>serum>whole blood. The micellar system provided some differences in the tissue distribution of 17-AAG into the brain, heart, and bladder, while similar concentrations to the standard formulation were found in other tissues analyzed. The tissue to serum ratio (Kₚ) values in all tissues (FIG. 4(b)) for the micellar formulation was lower than the control. The Kp values following administration of the standard formulation (CrEL-EtOH-PEG400) and for 17-AAG encapsulated in the micellar formulation (FIG. 4 lower panel) were statistically different for brain, heart, bladder and whole blood; indicative of some partitioning preference between tissues (FIG. 4(b)). The differences in Kp values between formulations among some tissues may be attributed to several factors, including the differences in disposition of the carrier, partitioning, clearance, and volume of distribution of 17-AAG between the control and the micellar formulations according to the invention.

### CONCLUSIONS

The lack of suitable formulations has hindered the progression of 17-AAG into clinical trials. Newer derivatives, such as 17-DMAG (alvespimycin), have overcome some problems associated with water solubility. However, the preferential and rapid clearance of these derivatives by the liver limits drug distribution into tumors, thereby severely limiting the efficacy of the drug. A formulation of 17-AAG that does not require organic co-solvents or harsh surfactants has been prepared. The formulation can solubilize 1.5 mg/mL of 17-AAG in PEG-PLA (12:6 kDa) micelles. A second formulation of 17-AAG that does not require organic co-solvents or surfactants has been prepared. This formulation can solubilize about five mg/mL of 17-AAG in PEG-PLA (2:2 kDa) micelles. Similar work with paclitaxel encapsulation into PEG-PLA micelles has demonstrated that this safer micellar formulation can minimize adverse side effects associated with CrEL following administration of the drug to patients. In addition, the nanoscale dimensions will further benefit tumor specificity of the drug through the EPR effect even in the absence of targeting ligands.

### Example 3

### Polymer Composition and Drug Loading

Certain specific micelles according to the invention were prepared as illustrated by the data in Tables 4 and 5.

TABLE 4
Various Drug:Polymer Ratios of Prepared PEG-PLA Micelles

| Drug: Polymer | | | | | | | |
|---|---|---|---|---|---|---|---|
| (wt./wt.) | PEG:PLA | PAX | AAG | | | AcCN | DW |
| (mg polymer) | (mw) | only | only | PAX + | AAG | (mL) | (mL) |
| 1:2.5 (5 mg) | 2k-2k | 2 mg | 2 mg | 2 mg | 2 mg | 0.5 mL | 0.5 mL |
| | 5k-3k | 2 mg | 2 mg | 2 mg | 2 mg | | |
| | 5k-6k | 2 mg | 2 mg | 2 mg | 2 mg | | |
| | 6k-12k | | | | | | |
| | 12k-3k | 2 mg | 2 mg | 2 mg | 2 mg | | |
| | 12k-6k | 2 mg | 2 mg | 2 mg | 2 mg | | |
| 1:5.0 (10 mg) | 2k-2k | 2 mg | 2 mg | 2 mg | 2 mg | 0.5 mL | 0.5 mL |
| | 5k-3k | 2 mg | 2 mg | 2 mg | 2 mg | | |
| | 5k-6k | 2 mg | 2 mg | 2 mg | 2 mg | | |
| | 6k-12k | | | | | | |
| | 12k-3k | 2 mg | 2 mg | 2 mg | 2 mg | | |
| | 12k-6k | 2 mg | 2 mg | 2 mg | 2 mg | | |
| 1:7.5 (15 mg) | 2k-2k | 2 mg | 2 mg | 2 mg | 2 mg | 0.5 mL | 0.5 mL |
| | 5k-3k | 2 mg | 2 mg | 2 mg | 2 mg | | |
| | 5k-6k | 2 mg | 2 mg | 2 mg | 2 mg | | |
| | 6k-12k | | | | | | |
| | 12k-3k | 2 mg | 2 mg | 2 mg | 2 mg | | |
| | 12k-6k | 2 mg | 2 mg | 2 mg | 2 mg | | |
| 1:10 (20 mg) | 2k-2k | 2 mg | 2 mg | 2 mg | 2 mg | 0.5 mL | 0.5 mL |
| | 5k-3k | 2 mg | 2 mg | 2 mg | 2 mg | | |
| | 5k-6k | 2 mg | 2 mg | 2 mg | 2 mg | | |
| | 6k-12k | | | | | | |
| | 12k-3k | 2 mg | 2 mg | 2 mg | 2 mg | | |
| | 12k-6k | 2 mg | 2 mg | 2 mg | 2 mg | | |

For each sample of micelles prepared in Table 4, 0.5 mL of acetonitrile (AcCN) was used to dissolve the drug and polymer (at ˜60° C.) before concentrating the solution and adding 0.5 mL of deionized water (DW) to form the micelles. The micelles were isolated by filtration using a 0.45 μm pore filter. For the paclitaxel/17-AAG dual agent micelles (PAX+AAG), the drug to polymer ratio was calculated by the mass of each drug. For example, 2 mg of paclitaxel and 2 mg of 17-AAG were solubilized with 5 mg of PEG-PLA Polymer (2K-2K) for the 1:2.5 drug:polymer ratio micelles (i.e., the ratio was calculated by the mass of one drug, not the sum of drugs).

TABLE 5
Micelle Drug Loading (mg/mL) (HPLC Analysis)

| | | Initial | | | | 1 day, RT (23° C.) | | | |
|---|---|---|---|---|---|---|---|---|---|
| | | PAX | AAG | | | PAX | AAG | | |
| | | only | only | PAX + | AAG | only | only | PAX + | AAG |
| 1:2.5 | 2k-2k | 1.41 | — | 0.24 | 0.23 | 0.93 | — | 0.16 | 0.14 |
| | 5k-3k | 3.78 | 1.57 | 2.40 | 3.25 | 0.63 | 1.39 | 0.97 | 1.31 |
| | 5k-6k | 3.31 | 2.53 | 3.76 | 4.11 | 0.69 | 2.11 | 2.78 | 3.30 |
| | 12k-3k | 2.93 | 1.51 | 0.45 | 0.51 | 0.20 | 0.83 | 0.15 | 0.19 |
| | 12k-6k | 2.73 | 1.58 | 3.64 | 3.74 | 0.21 | 1.16 | 2.30 | 2.35 |
| 1:5.0 | 2k-2k | | 3.86 | 3.56 | 3.71 | | 3.16 | 2.85 | 3.01 |
| | 5k-3k | 3.59 | 3.58 | 4.77 | 3.75 | 0.47 | 3.20 | 4.01 | 3.19 |
| | 5k-6k | 4.89 | 4.63 | 4.09 | 4.03 | 0.42 | 4.37 | 3.87 | 3.85 |
| | 12k-3k | 3.07 | 1.20 | 0.59 | 1.04 | 0.20 | 1.04 | 0.36 | 0.63 |
| | 12k-6k | 2.48 | 3.51 | 3.59 | 3.97 | 0.17 | 3.16 | 3.53 | 3.87 |
| 1:7.5 | 2k-2k | — | — | 3.77 | 4.18 | — | — | 3.24 | 3.56 |
| | 5k-3k | 3.13 | 3.56 | 4.06 | 4.68 | 0.46 | 3.38 | 3.70 | 4.23 |
| | 5k-6k | 3.39 | 4.04 | 4.39 | 4.67 | 0.27 | 3.78 | 4.19 | 4.44 |
| | 12k-3k | 3.46 | 2.79 | 3.79 | 3.96 | 0.20 | 2.22 | 3.23 | 3.38 |
| | 12k-6k | 3.66 | 3.88 | 3.50 | 4.65 | 0.19 | 3.82 | 3.39 | 4.52 |
| 1:10 | 2k-2k | 3.59 | — | 3.94 | 3.84 | 0.41 | — | 3.47 | 3.34 |
| | 5k-3k | 3.21 | 4.20 | 4.24 | 4.16 | 0.23 | 3.93 | 3.82 | 3.73 |
| | 5k-6k | 3.88 | 4.33 | 3.29 | 4.39 | 0.64 | 4.20 | 3.14 | 4.20 |
| | 12k-3k | 3.89 | 3.16 | 3.59 | 3.68 | 0.22 | 2.67 | 3.22 | 3.27 |
| | 12k-6k | 3.51 | 3.61 | 3.83 | 4.11 | 0.17 | 3.10 | 3.54 | 3.73 |

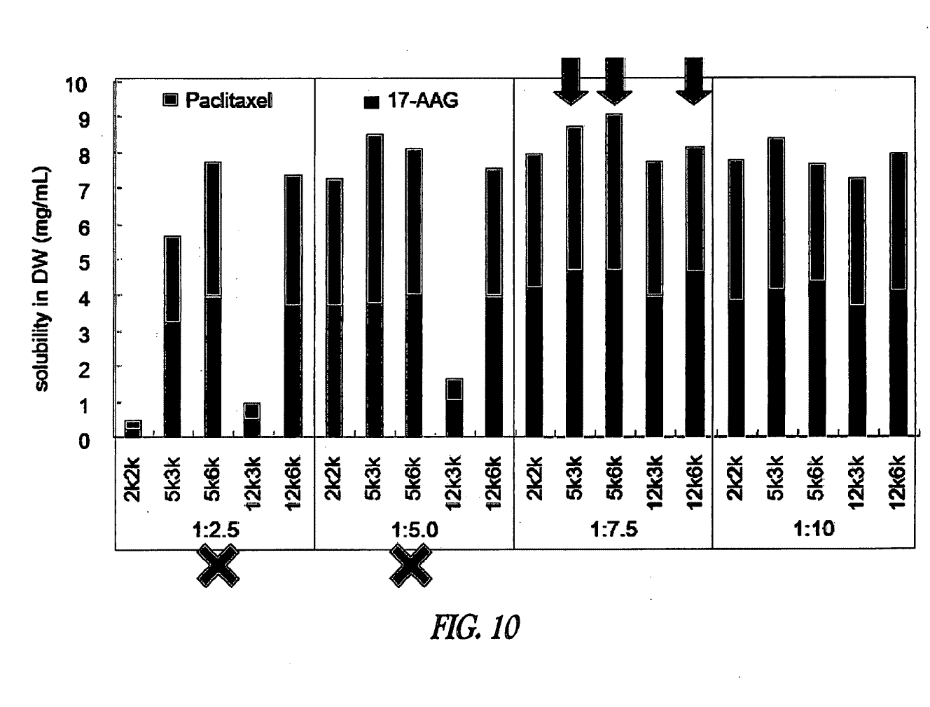

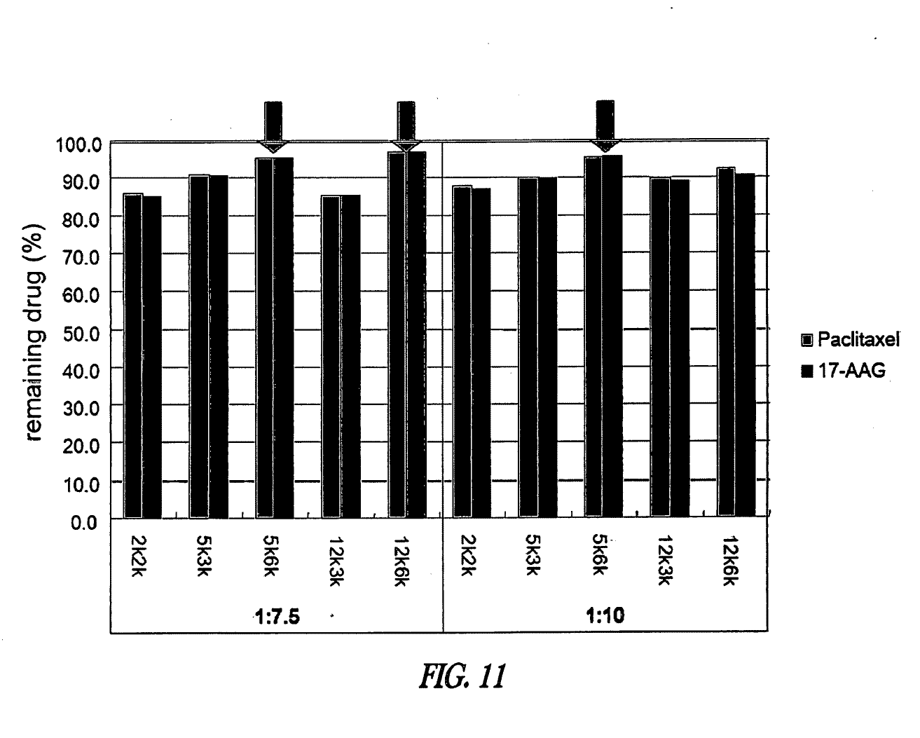

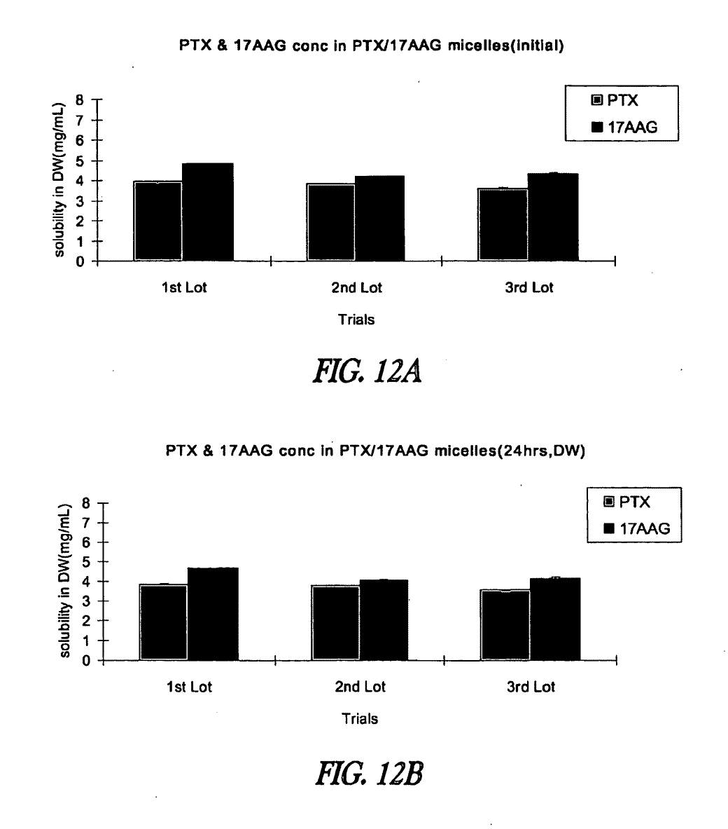

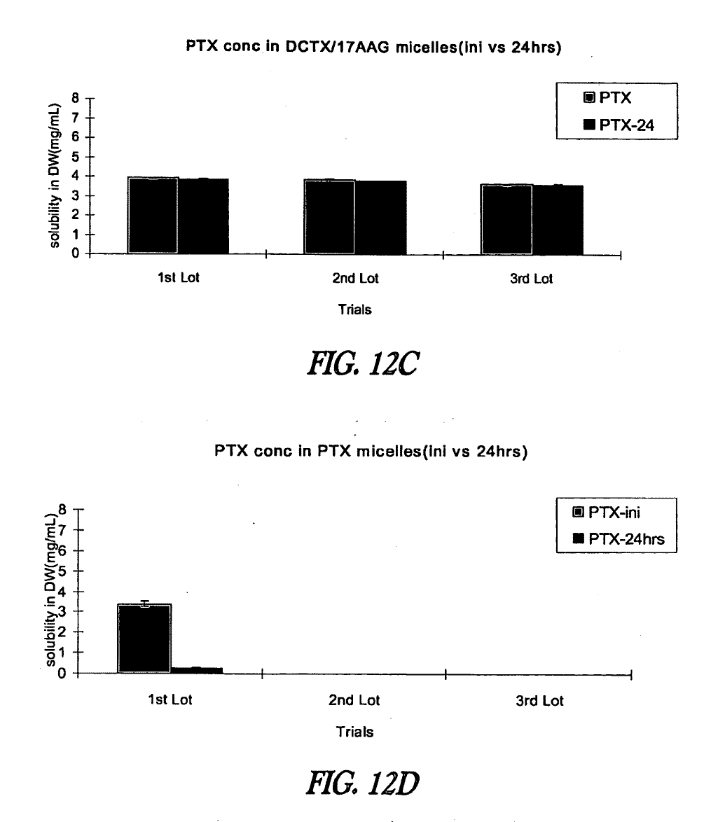

Table 5 illustrates the physical stability of various PEG-PLA micelles encapsulating paclitaxel (PAX), 17-AAG, or the two drugs loaded together. The values in the table indicate the amount of active agent retained by the micelles after 24 hours in distilled water storage at room temperature. Micelles that encapsulate 17-AAG, either alone or in combination with paclitaxel were significantly more stable the paclitaxel single agent micelles. Similar stability results were obtained for micelles containing combinations of 17-AAG/docetaxel or 17-AAG/etoposide (see FIGS. 12-15). FIG. 10 illustrates the amount of drug formulation that can be dissolved in deionized water (mg/mL).

PEG-PLA micelles with block sizes of 5K:6K (PEG:PLA, respectively) suitably solubilize either paclitaxel alone or 17-AAG alone at about 5 mg/mL. The paclitaxel micelles, however, lose the drug from the micelle core faster than 17-AAG micelles (over a period of 24 hours) by precipitation of paclitaxel from the micelles (e.g., loss of paclitaxel aqueous solubility). It was found that the 17-AAG micelles are significantly more stable and soluble than micelles containing certain other active agents alone (e.g., paclitaxel). Accordingly, 17-AAG can be considered a micelle stabilizer.

An unexpected result was found when both paclitaxel and 17-AAG were loaded at the same time into PEG-PLA micelles. In these micelles, the solubilization of each drug approached the level obtained when each drug was loaded by itself into PEG-PLA micelles. For example, in 5K:6K micelles, about 4-5 mg/mL loading was achieved for each of both drugs (compared to about 5 mg/mL for the drugs alone).

In addition to these unprecedented results, PEG-PLA micelles that contain both drugs in their cores were shown to be more stable with respect to the loss of the non-17-AAG active agent (see FIG. 11). It was discovered that an interaction between the active agents in the cores of PEG-PLA micelles, for example, 17-AAG and paclitaxel, increased the micelle stability. Polymeric micelles with 5K-6K and 12K-6K PEG-PLA showed added stability at a 1:7.5 drug:polymer ratio of micelles. The same result was found for micelles that included 17-AAG along with docetaxel or etoposide. Accordingly, combinations of 17-AAG (an Hsp90 inhibitor and a chemotherapeutic agent (e.g., paclitaxel, docetaxel, teniposide, and/or etoposide) can be administered without resorting to organic solvents, surfactants, and the like.

These results are further illustrated in FIGS. 10 and 11, which show results of the co-solubilization of 17-AAG and etoposide, docetaxel, and paclitaxel by PEG-PLA micelles (PEG-PLA 5K6K, Drug:Polymer ratio=1:7.5). Specifically, FIG. 10 shows the solubilization of paclitaxel and 17-AAG together in PEG-PLA micelles. Micelles prepared from 2K-2K and 12K-3K PEG-PLA provided lower drug solubilization results at below a 1:5 drug:polymer ratio. Micelles prepared from 5K-3K, 5K-6K, and 12K-6K PEG-PLA provided the highest solubilization at a 1:7.5 drug:polymer ratio.

Etoposide and docetaxel can be co-solubilized with 17-AAG in the same way as paclitaxel and 17-AAG. Also, more etoposide stays in solution over 24 hours in the presence of 17-AAG. FIGS. 12-15 illustrates optical density changes upon the precipitation of a second active agent (paclitaxel, docetaxel, or etoposide), due to certain amounts of degradation, including drug release from PEG-PLA micelles in water. Micelles containing 17-AAG prevent and/or inhibit this degradation from occurring.

FIGS. 12(a)-(d) illustrate the solubility of paclitaxel/17-AAG micelles and their stability after 24 hours (PEG-PLA 5K:6K, drug:polymer ratio=1:7.5). A Malvern Zetasizer was used for generating particle size data. FIG. 12(c) shows that more than 98% of PAX and more than 97% of the 17-AAG remained in the micelles after 24 hours. FIG. 12(d) shows that only 8% of PAX remained in the PAX-alone micelles after 24 hours, indicating that the presence of 17-AAG provides significant stability to the physically mixed micelles.

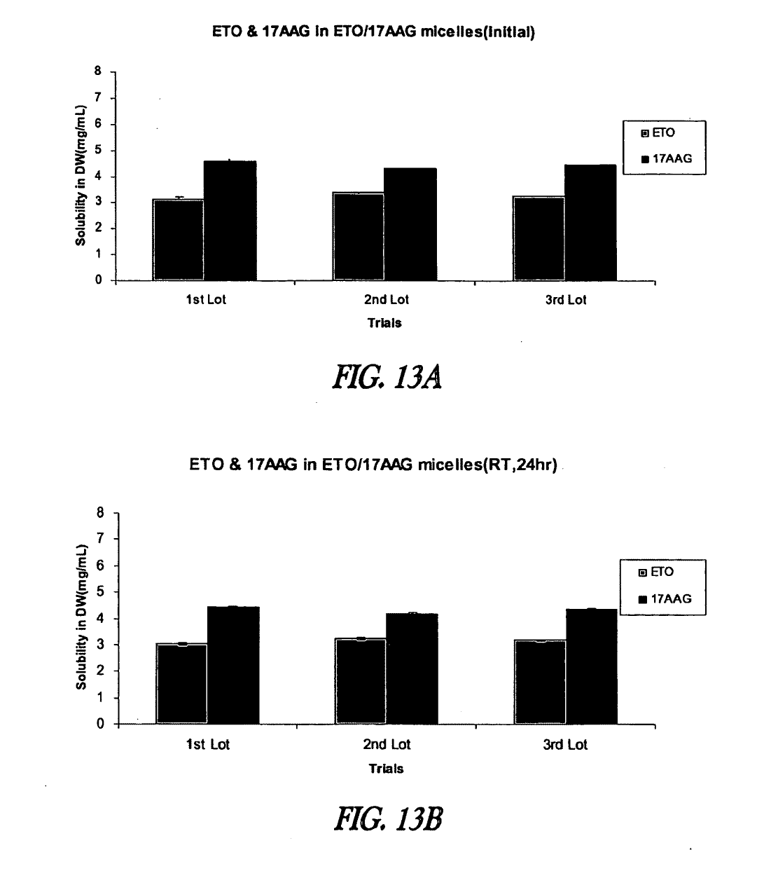

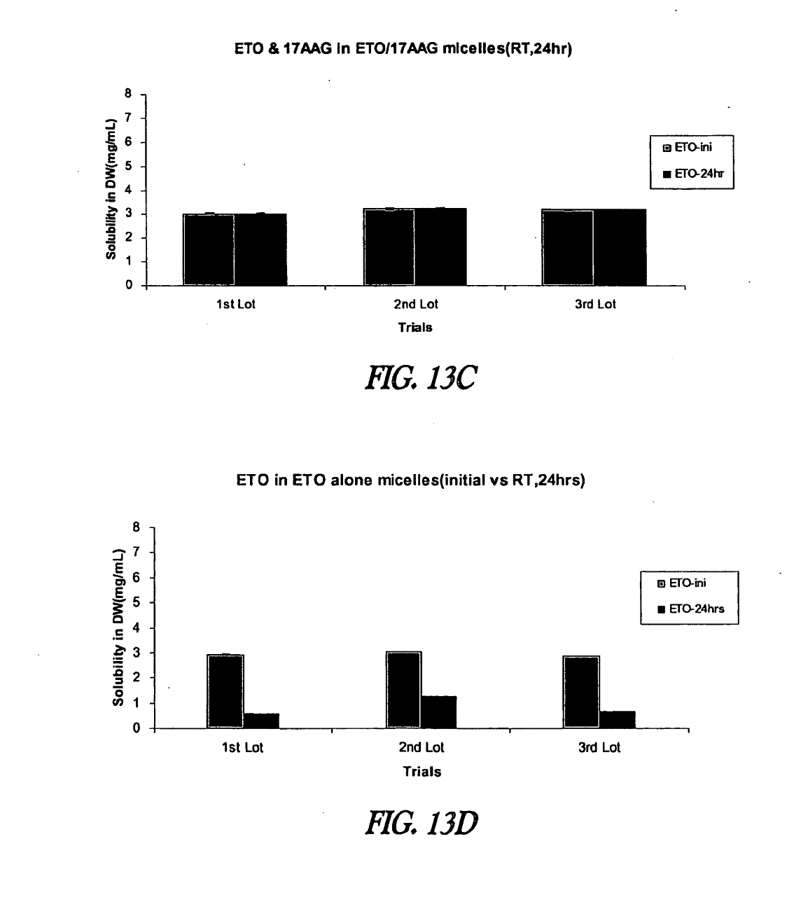

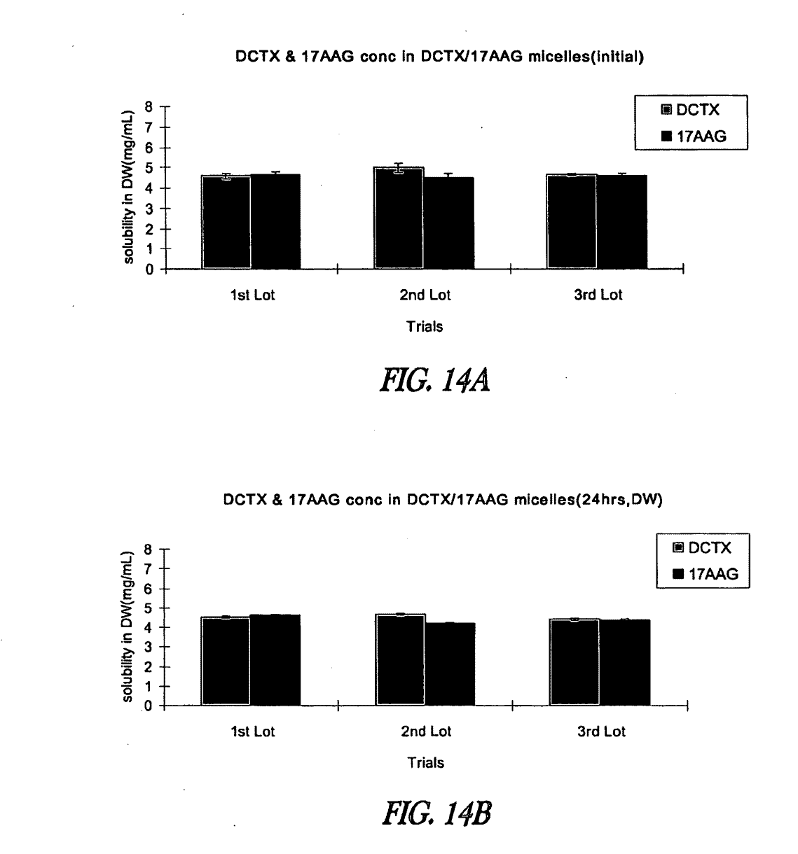

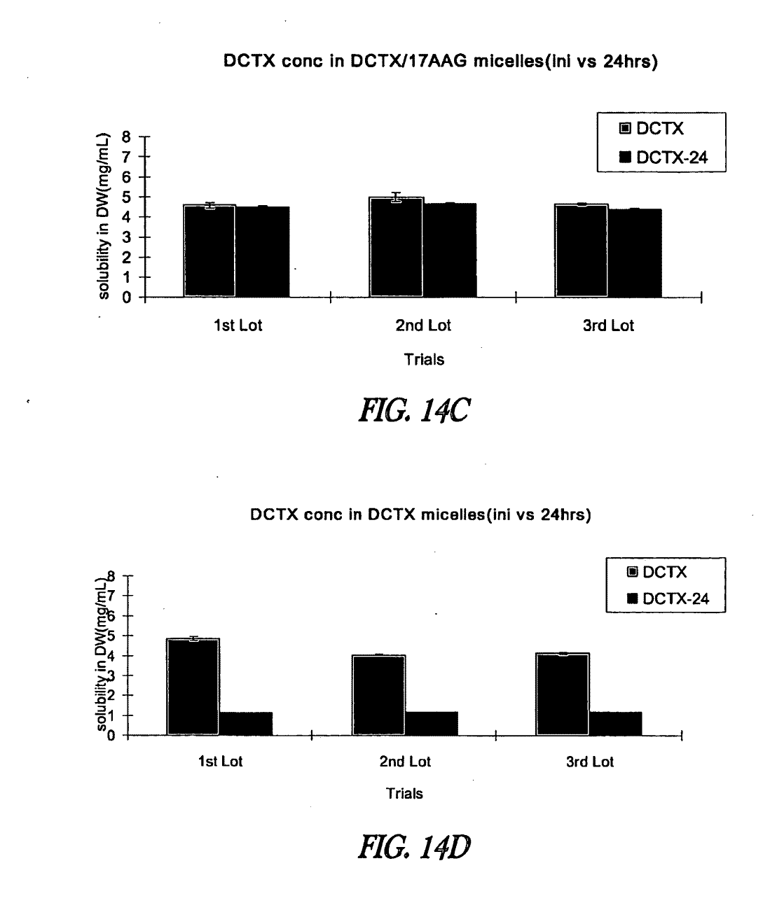

Similar results were observed for etoposide (ETO) and etoposide/17-AAG micelles (FIGS. 13(a)-(d)) and docetaxel (DCTX) and docetaxel/17-AAG micelles (FIGS. 14(a)-(d)). Micelles prepared from PEG-PLA 5K:6K were used to prepare the data for FIGS. 13 and 14, with drug:polymer ratios of 1:7.5 (wt./wt.). FIG. 13(c) shows that nearly 100% of etoposide and 17-AAG remained in the micelles after 24 hours, while significant loss of etoposide was observed in etoposide-alone micelles (FIG. 13(d): over 70% average loss (n=3)). The etoposide began to precipitate from the etoposide-alone micelles after only 1-2 hours.

FIG. 14(c) shows that the inclusion of 17-AAG in micelles with docetaxel provided a significant increase in stability over docetaxel-alone micelles. Over 95% of both the docetaxel and 17-AAG remained in the micelles after 24 hours, while significant loss of docetaxel was observed in docetaxel-alone micelles (FIG. 14(d): over 73.2% average loss (n=3)). The docetaxel began to precipitate from the docetaxel-alone micelles after only 2 hours.

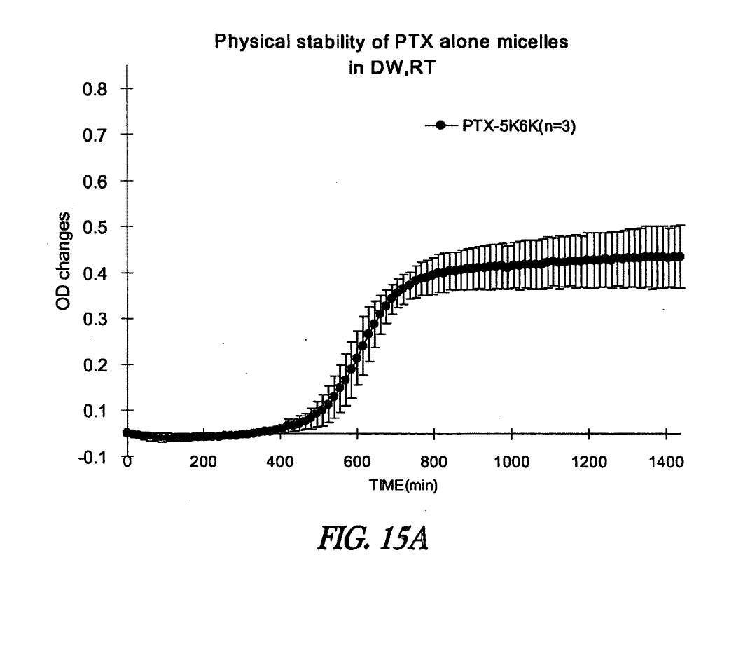

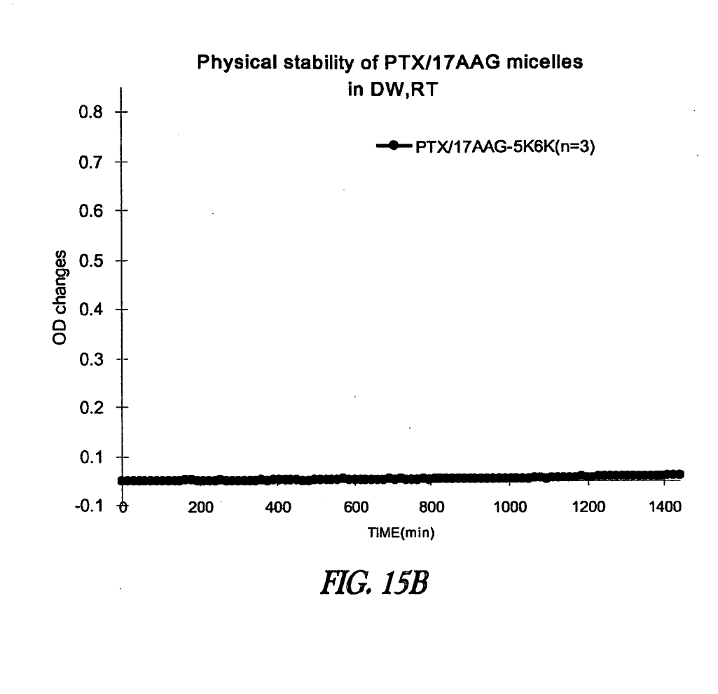

FIGS. 15(a) and 16(b) further illustrate the additional stability provided to micelles when 17-AAG is incorporated into micelles, in addition to paclitaxel. Measurements of optical density (OD) changes in the drug-micelles were made at 650 nm(RT) using a Varian® Cary 50 with a dip probe (0 to 1440 minutes; every 15 minutes; acquisition: 0.1 seconds). A 1:7.5 drug:polymer ratio was used with 5K:6K PEG-PLA micelles. Paclitaxel encapsulated micelles were loaded with 13.4% paclitaxel (wt./wt.) and paclitaxel/17-AAG dual-agent micelles were loaded at 26.8% drug loading (combined; 13.4% each drug). FIG. 15(a) shows that significant amounts of paclitaxel were lost from the paclitaxel encapsulated micelles, while paclitaxel/17-AAG micelles were substantially stable over 24 hours (FIG. 15(b)).

Further unexpected results were observed when simply mixed micelle compositions were prepared. For example, PEG-PLA micelles were prepared where a sample of micelles encapsulated paclitaxel and a second sample of micelles encapsulated 17-AAG. When the two samples of micelles were combined into a single aqueous formulation, the paclitaxel single agent micelles were more stable against precipitation than a purely paclitaxel single agent formulation of PEG-PLA micelles. Thus, the paclitaxel encapsulated micelles in a formulation with 17-AAG micelles had increased stability compared to a formulation made of only paclitaxel incorporated PEG-PLA micelles. In some embodiments, equilibration of active agents may take place between micelles in a formulation. Other single agent micelles can be similarly stabilized by forming simply mixed formulations with 17-AAG encapsulated PEG-PLA micelles.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A composition comprising 17-AAG-encapsulated micelles, wherein the micelles comprise poly(ethylene glycol)-block-poly(lactic acid) polymers;
the hydrophobic poly(lactic acid) block of the polymers orient toward the interior of each micelle, and the hydrophilic poly(ethylene glycol) block of the polymers orient toward the exterior of each micelle;
the molecular weight of the poly(ethylene glycol) block is about 1,000 to about 35,000 g/mol and the molecular weight of the poly(lactic acid) block is about 1,000 to about 15,000 g/mol;
the drug loading of the 17-AAG in the micelles is about 1 wt. % to about 25 wt. % with respect to the weight of the micelles; and
the composition is substantially free of ethanol, dimethyl sulfoxide, castor oil, and castor oil derivatives.

2. The composition of claim 1 wherein the drug loading of the 17-AAG in the micelles is 10 wt. % to about 20 wt. %.

3. The composition of claim 1 further comprising and aqueous vehicle, wherein the concentration of the 17-AAG is about 0.6 mg/mL to about 20 mg/mL, with respect to the volume of the aqueous vehicle.

4. (canceled)

5. The composition of claim 1 wherein the composition comprises less than about 2 wt. % of ethanol, dimethyl sulfoxide, castor oil, and castor oil derivatives.

6. The composition of claim 1 wherein the molecular weight of the poly(ethylene glycol) block is about 10,000 to about 14,000 g/mol and the molecular weight of the poly(lactic acid) block is about 5,000 to about 7,000 g/mol, and the average diameter of the micelles is about 250 nm to about 270 nm.

7. The composition of claim 1 wherein the molecular weight of the poly(ethylene glycol) block is about 1,500 to about 2,500 g/mol and the molecular weight of the poly(lactic acid) block is about 1,500 to about 2,500 g/mol, and the average diameter of the micelles is about 40 nm to about 60 nm.

8. The composition of claim 1, further comprising a second active agent encapsulated in one or more PEG-PLA micelles, and the drug loading of the second active agent in the micelles is about 1 wt. % to about 25 wt. %, with respect to the weight of the micelles.

9. The composition of claim 8 wherein the 17-AAG and the second active agent are incorporated together into one or more PEG-PLA micelles.

10. The composition of claim 8 wherein the 17-AAG and the second active agent are incorporated individually into PEG-PLA micelles, and the micelles are combined in a single aqueous vehicle.

11. The composition of claim 8 wherein the second active agent is paclitaxel, docetaxel, etoposide, or teniposide.

12. A pharmaceutical composition comprising the composition of claim 1 and an aqueous carrier, wherein the composition is formulated for intravenous or intraperitoneal administration and the aqueous carrier comprises saline or a carbohydrate.

13. A method of administering 17-AAG to a patient that has, or has been diagnosed with, a condition that can be treated by administration of 17-AAG, comprising administering an effective amount of a composition comprising 17-AAG-encapsulated micelles, wherein the micelles comprise poly(ethylene glycol)-block-poly(lactic acid) polymers;
the hydrophobic poly(lactic acid) block of the polymers orient toward the interior of each micelle, and the hydrophilic poly(ethylene glycol) block of the polymers orient toward the exterior of each micelle;
the molecular weight of the poly(ethylene glycol) block is about 1,000 to about 35,000 g/mol and the molecular weight of the poly(lactic acid) block is about 1,000 to about 15,000 g/mol;
the drug loading of the 17-AAG in the micelles is 1 wt. % to about 25 wt. % with respect to the weight of the micelles; and
the composition is substantially free of ethanol, dimethyl sulfoxide, castor oil, and castor oil derivatives.

14. The method of claim 13 wherein the 17-AAG in the micelles is administered in an amount of about 4 mg/m² to about 4000 mg/m².

15. (canceled)

16. (canceled)

17. The method of claim 13 wherein the composition further comprises a second active agent within the micelles that contain the 17-AAG.

18. A method of inhibiting or killing cancer cells comprising contacting the cells with an effective inhibitory or lethal amount of a composition of claim 1.

19. (canceled)

20. (canceled)

21. The method of claim 18 wherein the cancer cells are human breast cancer cells, human prostate cancer cells, or leukemia cells.

22. A method of inhibiting Hsp 90 comprising contacting the Hsp 90 with an effective inhibitory amount of a composition of claim 1.

23. (canceled)

24. (canceled)

25. A method of increasing the half-life of 17-AAG in the blood of a mammal comprising administration of the composition of claim 1 , in comparison to the half-life of 17-AAG in the blood after administration of 17-AAG with a carrier that does not comprise micelles formed from poly(ethylene glycol)-block-poly(lactic acid) polymers.

26. A method of preparing 17-AAG-encapsulated micelles comprising dissolving 17-AAG and poly(ethylene glycol)-block-poly(lactic acid) polymeric units in a water-miscible solvent, adding the solution to a dialysis bag, adding the dialysis bag to a water-containing bath to provide the 17-AAG-encapsulated micelles in the dialysis bag, and isolating the micelles.

27-30. (canceled)

31. A method of preparing 17-AAG-encapsulated micelles comprising dissolving 17-AAG and poly(ethylene glycol)-block-poly(lactic acid) polymer chains in a water-miscible solvent to provide a solution, removing the water-miscible solvent to provide a solid or oil-like composition comprising the polymer and the 17-AAG, adding water to the composition to provide an aqueous mixture, centrifuging the mixture to provide a supernatant containing the 17-AAG-encapsulated micelles and a sediment, and removing the sediment, to provide isolated 17-AAG-encapsulated micelles.
